(19) United States
(12) Patent Application Publication
(10) Pub. No.: US 2026/0284019 A1
(43) Pub. Date: Sep. 24, 2026

(54) CARBOXAMIDE COMPOUNDS AS SUMO ACTIVATING COMPOUNDS FOR TREATMENT OF CARDIOVASCULAR DISEASES

(71) Applicant: NOVOHEART INTERNATIONAL LIMITED, Kowloon (HK)

(72) Inventors: Andy Wong, Kowloon (HK); Ronald A. Li, Pok Fu Lam (HK); Roger J. Hajjar, Boston, MA (US)

(21) Appl. No.: 19/474,525

(22) PCT Filed: Apr. 12, 2024
(86) PCT No.: PCT/IB2024/053629

§ 371 Date: Oct. 10, 2025

Related U.S. Application Data
(60) Provisional application No. 63/459,063, filed on Apr. 13, 2023.

(51) Int. Cl. A61K 31/437; A61K 31/444; A61K 31/4709

(52) U.S. Cl. CPC A61K 31/437; A61K 31/444; A61K 31/4709

(57) ABSTRACT

Disclosed are carboxamide derivative compounds effective as therapeutics in treating heart disease such as heart failure, cardiomyopathy, diastolic dysfunction, and the heart disease aspect of other diseases or disorders, such as muscular dystrophy. Also disclosed are methods of treating subjects with such diseases and methods of screening for compounds effective as therapeutic in treating such diseases. The screening methods provide rapid ex vivo approaches to identifying therapeutics that involve the use of cardiac tissue strips, polymeric substrates such as polydimethylsiloxane that have posts with curved features to retain tissues and measure contractile force of cardiomyocyte tissue strips, and/or one or more cardiac organoids in chambered devices to facilitate manipulation and monitoring.

### FIELD

The disclosure relates to the field of medicine and to the field of devices, compounds, and methods useful in culturing, maintaining, monitoring or testing organoids and tissues, and in the treatment of heart disease.

### BACKGROUND

Twenty-six million people suffer from heart failure (HF) in the world, with 6.2 million of these people being patients in the United States. Of all the cardiovascular diseases, HF is the only heart disease being diagnosed with increasing incidence and prevalence. While a number of treatment strategies have currently been used for the treatment of various forms of HF, the increasing incidence and prevalence of the disease highlights the critical need for novel targets and treatment strategies. Decreased activity and expression of the cardiac sarcoplasmic reticulum calcium ATPase (SERCA2a), a critical pump regulating calcium cycling in cardiomyocyte, are hallmarks of heart failure. The small ubiquitin-like modifier type 1 (SUMO-1) is a regulator of SERCA2a.

N1061 (N-(4-methoxybenzo[d]thiazol-2-yl)-5-(4-methoxyphenyl)-1,3,4-oxadiazol-2-amine) is the only first-in-class SUMO-1 activator that has been reported to SUMOylate SERCA2a in treating heart diseases. The half-life of N106 in vivo is too short to provide effective clinical treatment of heart disease, however. Thus, a need continues to exist in the art for products that modulate the activity of SERCA2a, for example by SUMOylation, in the treatment of heart disease such as heart failure.

Heart failure (HF) affects 26 million people around the world, 6.2 million of which are in the United States)². HF diagnosis continues to increase in both incidence and prevalence³, highlighting the need for novel targets and treatment strategies. The cardiac sarcoplasmic reticulum Ca2+-ATPase (SERCA2a) is a key pump responsible for intracellular calcium handling and contractility in cardiac cells. Impaired calcium reuptake resulting from decreased expression and activity of SERCA2a is a hallmark of HF. SERCA2a gene transfer has been shown to restore cardiac energetics^{4,5}, decrease ventricular arrhythmias^{9,10}, block smooth muscle cell proliferation, and enhance coronary flow through the activation of nitric oxide synthase in endothelial cells⁶. Changes in posttranslational modifications (PTMs) of SERCA2a render it dysfunctional, and restoration of SERCA2a by gene transfer does not abrogate the PTMs of the transporter. Therefore, SERCA2a's enzymatic dysfunction, in addition to its decreased expression, needs to be addressed in the failing heart, to normalize calcium cycling.

SUMOylation, the covalent attachment of Small Ubiquitin-like MOdifier (SUMO) polypeptides to target proteins, is among the most important post-translational modifications that regulate the functional properties of a large number of proteins. SUMOylation, the attachment of a SUMO protein to another protein, is carried out in sequential steps catalyzed by three enzymes: E1 (heterodimer composed of SUMO-activating enzyme 1 (i.e., SAE1) and SUMO-activating enzyme 2 (i.e., SAE2)) activating enzyme, E2 ubiquitin-conjugating enzyme 9 (Ubc9) conjugating enzyme, and E3 SUMO ligase enzyme⁷⁻⁹. In the heart, the level and activity of SERCA2a in cardiomyocytes are modulated in parallel with the levels of small ubiquitin-like modifier type 1 (SUMO-1). SUMO-1 gene transfer leads to restoration of SERCA2a levels, improved hemodynamic performance, and reduced mortality in a murine model of pressure overload-induced HF¹⁰.

Inhibition of the various SUMO pathway components to reduce protein SUMOylation has been targeted as a strategy for various diseases including cancer, viral infection, and cystic fibrosis^{12, 13}. In contrast, there are few reports of activation of SUMO conjugation. Reported activators of SUMOylation have been shown to be neuroprotective in an in vitro model of ischemia. Studies have demonstrated that protein SUMOylation is associated with critical cellular pathways^{14, 15}. It has also been demonstrated that SUMOylation activators for the substrate protein SERCA2a¹⁰ leads to the subsequent activation of SERCA2a and improved muscle contraction.

### SUMMARY

Disclosed herein is a new series of compounds that activate E1 Ligase activity in biochemical assays independently from first generation compounds like N106. The disclosed series of compounds were tested for effects on engineered cardiac tissues generated by human pluripotent stem cell (hPSC)-derived human ventricular cardiomyocytes (hvCMs). Activated E1 Ligase in turn activates the SUMO-1 protein in cardiomyocytes, leading to increased SUMOylation and activity of SERCA2a in these cells, which has a beneficial effect in patients experiencing heart failure. The engineered cardiac tissues were assembled in the form of human ventricular cardiac tissue strips (hvCTS) and exposed to each of the series of compounds disclosed herein. The results showed an increase in developed force and an improvement in contractile kinetics in the hvCTS. The disclosed methods are useful in screening for candidate cardiac therapeutics and the identified compounds are expected to provide effective treatments for heart failure.

Gene transfer of SUMO-1 in rodents and large animal models of heart failure restores cardiac function. The data disclosed herein establish that small-molecule activator-targeting SERCA2a SUMOylation shows promise as a therapeutic strategy for treatment of heart failure. Disclosed herein are small molecules that are efficacious in activating SUMO-1, thereby increasing SERCA2a activity as a novel treatment for heart failure. In particular, 11 carboxamide derivative compounds are disclosed. These carboxamide derivatives each have two chiral centers, as shown by the dotted circles highlighting the base carboxamide compound in FIG. 1A. The chiral centers result in each compound existing as any one of four stereoisomers. As described below, FIGS. 2A-K presents the two-dimensional chemical structure of each of the 11 carboxamide derivative compounds, which differ from the base carboxamide compound in the substituents attached to the phenyl group. Because the substituents do not affect either of the two chiral centers, the four stereoisomers of each of the 11 carboxamide derivative compounds can be defined using the same convention. Using the base compound to define the naming convention, the S-S stereoisomer is designated stereoisomer 1, the R-S stereoisomer is stereoisomer 2, the S-R stereoisomer is stereoisomer 3, and the R-R stereoisomer is stereoisomer 4, as shown in FIGS. 1B-1E, respectively. Thus, for example, carboxamide derivative compound 6, having the 2-D structure shown in FIG. 2F, exists as any of the following four stereoisomers: 1-(4-benzoylphenyl)-(R)-5-oxo-N—((R)-1-(pyrazolo[1,5-a]pyridin-3-yl)ethyl) pyrrolidine-3-carboxamide, which is stereoisomer 6-1, 1-(4-benzoylphenyl)-(S)-5-oxo-N—((S)-1-(pyrazolo[1,5-a]pyridin-3-yl)ethyl) pyrrolidine-3-carboxamide, which is stereoisomer 6-2, 1-(4-benzoylphenyl)-(R)-5-oxo-N—((S)-1-(pyrazolo[1,5-a]pyridin-3-yl)ethyl) pyrrolidine-3-carboxamide, which is stereoisomer 6-3, and 1-(4-benzoylphenyl)-(S)-5-oxo-N—((R)-1-(pyrazolo[1,5-a]pyridin-3-yl)ethyl) pyrrolidine-3-carboxamide, which is stereoisomer 6-4.

In one aspect, the disclosure provides a method of treating heart disease comprising administering a therapeutically effective amount of a carboxamide derivative compound to a patient with heart disease, wherein the carboxamide derivative compound induces increased SUMOylation of sarcoplasmic reticulum calcium ATPase type 2a and/or induces increased E1 ligase activation of sarcoplasmic reticulum calcium ATPase type 2a. In some embodiments, the carboxamide derivative compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide). In some embodiments, the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide). In some embodiments, the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide). In some embodiments, the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide). In some embodiments, the heart disease is heart failure, cardiomyopathy, diastolic dysfunction, or muscular dystrophy. In some embodiments, the heart failure is pressure overload-induced heart failure or ischemic heart failure. In some aspects, the patient is a human patient.

Another aspect of the disclosure is drawn to a method of inducing a positive inotropic effect and/or lusitropic effect in a cardiomyocyte tissue by contacting the cardiomyocyte with a carboxamide derivative compound as described herein. In some embodiments the carboxamide derivative compound induces increased SUMOylation and/or increased E1 ligase activation of sarcoplasmic reticulum calcium ATPase type 2a. In some embodiments, the compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide). In some embodiments, the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide). In some embodiments, the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide). In some embodiments, the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide). In some embodiments, the a positive inotropic effect and/or lusitropic effect is increased force of cardiac contraction or increased frequency of beating of the cardiomyocyte tissue. In some embodiments, the compound is compound 6-1 (1-(4-benzoylphenyl)-(S)-5-oxo-N—((S)-1-(pyrazolo[1,5-a]pyridin-3-yl)ethyl) pyrrolidine-3-carboxamide), compound 6-2 (1-(4-benzoylphenyl)-(R)-5-oxo-N—((S)-1-(pyrazolo[1,5-a]pyridin-3-yl)ethyl) pyrrolidine-3-carboxamide), or compound 9-3 ((S)-5-oxo-N—((R)-1-(pyrazolo[1,5-a]pyridin-3-yl)ethyl)-1-(pyridin-3-yl) pyrrolidine-3-carboxamide). In some embodiments, a positive inotropic effect and/or lusitropic effect is increased force of cardiac contraction or increased frequency of beating of the cardiomyocyte tissue. In some aspects, a positive inotropic effect and/or lusitropic effect is increased force of cardiac contraction, improved cardiac contraction, improved relaxation kinetics, and/or increased frequency of beating of the cardiomyocyte tissue. In some aspects, a positive inotropic effect and/or lusitropic effect is increased contractile force, heartbeat frequency, and/or heartbeat dependent contractile force.

Yet another aspect of the disclosure is directed to a method of screening for a compound inducing an increase in contractile force, relaxation kinetics, heartbeat frequency, and/or heartbeat dependent contractile force in a human cardiomyocyte tissue comprising (a) contacting a cardiomyocyte tissue with at least one positive amount of the compound; (b) electrically stimulating the tissue; (c) measuring the contractile force, relaxation kinetics, heartbeat frequency, and/or heartbeat dependent contractile force of the cardiomyocyte tissue; (d) comparing the contractile force, relaxation kinetics, heartbeat frequency, and/or heartbeat dependent contractile force measured in (c) to the same property or properties of contractile force, relaxation kinetics, heartbeat frequency, and/or heartbeat dependent contractile force in a control cardiomyocyte tissue not contacted by the compound; and (e) identifying the compound as a compound inducing an increase in contractile force, relaxation kinetics, heartbeat frequency, and/or heartbeat dependent contractile force in a human cardiomyocyte if the contractile force, relaxation kinetics, heartbeat frequency, and/or heartbeat dependent contractile force increases with increasing amount of compound. In some embodiments, the cardiomyocyte tissue is a mature human ventricular cardiomyocyte tissue strip. In some embodiments, the compound is a carboxamide derivative compound. In some embodiments, the carboxamide derivative compound induces increased SUMOylation and/or E1 ligase activation of sarcoplasmic reticulum calcium ATPase type 2a. In some embodiments, the cardiomyocyte tissue is a mature human ventricular cardiomyocyte tissue strip. In some embodiments, the compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide). In some embodiments, the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide). In some embodiments, the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide). In some embodiments, the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

Another aspect of the disclosure includes a method of increasing the activity of at least one small ubiquitin-like modifier type 1 polypeptide in a cardiomyocyte comprising administering an effective amount of a carboxamide derivative compound as described herein. In some embodiments, the carboxamide derivative compound induces increased SUMOylation and/or E1 ligase activation of sarcoplasmic reticulum calcium ATPase type 2a. In some embodiments, the carboxamide derivative compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide). In some embodiments, the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide). In some embodiments, the compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide). In some embodiments, the compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

Yet another aspect of the disclosure is drawn to a method of increasing the activity of the sarcoplasmic reticulum calcium ATPase type 2a protein in a cardiomyocyte comprising administering an effective amount of a carboxamide derivative compound as described herein. In some embodiments, the carboxamide derivative compound induces increased SUMOylation and/or E1 ligase activation of sarcoplasmic reticulum calcium ATPase type 2a. In some embodiments, the carboxamide derivative compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide). In some embodiments, the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide). In some embodiments, the compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide). In some embodiments, the compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

Still another aspect of the disclosure is directed to a method of increasing the activity of E1 ligase in a cardiomyocyte comprising contacting the cardiomyocyte with an effective amount of a carboxamide derivative compound as described herein. In some embodiments, the carboxamide derivative compound induces increased SUMOylation and/or E1 ligase activation of sarcoplasmic reticulum calcium ATPase type 2a. In some embodiments, the carboxamide derivative compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide). In some embodiments, the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide). In some embodiments, the compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide). In some embodiments, the compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

In some embodiments, the cardiomyocyte is any mammalian cardiomyocyte cell type, including a primate, horse, cow, goat, sheep, zebrafish, pig, dog, rat, mouse, or a human cardiomyocyte cell type, e.g., a human ventricular cardiomyocyte cell type. In some aspects, the cardiomyocyte is ex vivo. In some aspects, the cardiomyocyte is in vivo. In some aspects, the cardiomyocyte is in a subject. In some aspects, the subject is a mammal. In some aspects, the mammal is a human subject.

Other features and advantages of the disclosure will become apparent from the following detailed description, including the drawings. It should be understood, however, that the detailed description and the specific examples, while indicating embodiments, are provided for illustration only, because various changes and modifications within the spirit and scope of the disclosure will become apparent to those skilled in the art from the detailed description.

### BRIEF DESCRIPTION OF THE DRAWINGS

FIGS. 1A-E. Depicted in FIG. 1A is the structure of a carboxamide compound (referenced herein as the base compound), i.e., 5-oxo-1-phenyl-N-(1-(pyrazolo[1,5-a]pyridin-3-yl)ethyl) pyrrolidine-3-carboxamide, which is therapeutically effective against cardiac diseases in the form of heart failure, cardiomyopathy, and diastolic dysfunction muscular dystrophy. The structure of the base compound has two chiral centers resulting in four stereoisomers (two pairs of enantiomers), which are resolvable by HPLC fractionation. FIG. 1B shows (R)-5-oxo-1-lphenyl-N—((R)-1-(pyrazolo[1,5-a]pyridin-3-yl)ethyl) pyrrolidine-3-carboxamide, which is stereoisomer 1 (i.e., Exemplary Compound 1-1) of the base compound. FIG. 1C shows(S)-5-oxo-1-phenyl-N—((S)-1-(pyrazolo[1,5-a]pyridin-3-yl)ethyl) pyrrolidine-3-carboxamide, which is stereoisomer 2 (i.e., Exemplary Compound 1-2) of the base compound. FIG. 1D shows (R)-5-oxo-1-phenyl-N—((S)-1-(pyrazolo[1,5-a]pyridin-3-yl)ethyl) pyrrolidine-3-carboxamide, which is stereoisomer 3 (i.e., Exemplary Compound 1-3) of the base compound. FIG. 1E shows(S)-5-oxo-1-phenyl-N—((R)-1-(pyrazolo[1,5-a]pyridin-3-yl)ethyl) pyrrolidine-3-carboxamide, which is stereoisomer 4 of the base compound.

FIGS. 2A-K show the structures of 11 exemplary carboxamide derivatives useful in the treatment of cardiac disease. The 11 exemplary derivatives are based on the Base Compound carboxamide (i.e., 5-oxo-1-phenyl-N-(1-(pyrazolo[1,5-a]pyridin-3-yl)ethyl) pyrrolidine-3-carboxamide) shown in FIG. 1A. The derivatives have various substituents attached to the phenyl group as shown in FIG. 2A-K. FIG. 2A shows 5-oxo-1-phenyl-N-(1-(pyrazolo[1,5-a]pyridin-3-yl)ethyl) pyrrolidine-3-carboxamide, i.e., the base compound, which may also be referenced as Exemplary Compound 1. FIG. 2B shows 1-(3-bromophenyl)-5-oxo-N-(1-(pyrazolo[1,5-a]pyridin-3-yl)ethyl) pyrrolidine-3-carboxamide, which may also be referenced as Exemplary Compound 2. FIG. 2C shows 1-(4-bromo-2-methylphenyl)-5-oxo-N-(1-(pyrazolo[1,5-a]pyridin-3-yl)ethyl) pyrrolidine-3-carboxamide, which may also be referenced as Exemplary Compound 3. FIG. 2D shows 1-(3,4-dimethoxyphenyl)-5-oxo-N-(1-(pyrazolo[1,5-a]pyridin-3-yl)ethyl) pyrrolidine-3-carboxamide, which may also be referenced as Exemplary Compound 4. FIG. 2E shows 1-(3,5-dimethoxyphenyl)-5-oxo-N-(1-(pyrazolo[1,5-a]pyridin-3-ylmethyl) pyrrolidine-3-carboxamide, which may also be referenced as Exemplary Compound 5. FIG. 2F shows 1-(4-benzoylphenyl)-5-oxo-N-(1-(pyrazolo[1,5-a]pyridin-3-yl)ethyl) pyrrolidine-3-carboxamide, which may also be referenced as Exemplary Compound 6. FIG. 2G shows 1-([1,1′-biphenyl]-4-yl))-5-oxo-N-(1-(pyrazolo[1,5-a]pyridin-3-yl)ethyl) pyrrolidine-3-carboxamide, which may also be referenced as Exemplary Compound 7. FIG. 2H shows 1-(4-hydroxyphenyl)-5-oxo-N-(1-(pyrazolo[1,5-a]pyridin-3-yl)ethyl) pyrrolidine-3-carboxamide, which may also be referenced as Exemplary Compound 8. FIG. 2I shows 5-oxo-N-(1-(pyrazolo[1,5-a]pyridin-3-yl)ethyl)-1-(pyridin-3-yl) pyrrolidine-3-carboxamide, which may also be referenced as Exemplary Compound 9. FIG. 2J shows 1-(5-methylpyridin-3-yl)-5-oxo-N-(1-(pyrazolo[1,5-a]pyridin-3-yl)ethyl) pyrrolidine-3-carboxamide, which may also be referenced as Exemplary Compound 10. FIG. 2K shows 5-oxo-N-(1-(pyrazolo[1,5-a]pyridin-3-yl)ethyl)-1-(quinolin-3-yl) pyrrolidine-3-carboxamide, which may also be referenced as Exemplary Compound 11.

FIGS. 3A-B shows a flowchart that depicts the compound screening strategy used to identify compounds that have positive effect on treatment of cardiomyopathy or heart failure. FIG. 3A provides a strategy for the biochemical assay of compounds starting with the E1 ligase assay of racemic (4 stereoisomers) mixtures of compounds for increased SUMO E1 Ligase activity. Compounds with positive effects are resolved into their pure stereoisomers and each stereoisomer is subjected to E1 ligase assay as well as functional analysis in engineered cardiac tissues. FIG. 3B shows functional analysis of the compounds in engineered cardiac tissues beginning with treatment of mature human ventricular cardiac tissue strips (hvCTS) with the pure stereoisomers. Stereoisomers with positive inotropic effects and/or lusitropic effects in hvCTS are tested for SERCA2a SUMOylation by western blotting. Stereoisomers with positive effects are further subjected to the same assays in hvCTS models of cardiomyopathy and heart failure.

FIG. 4A-B. FIG. 4A shows the effect of racemic mixtures of each of the 11 exemplary compounds of interest (see the brief description of FIGS. 2A-K for IUPAC names of the exemplary compounds) in SUMO E1 assays with luminescence measurements. Compounds 6, 9 and 11 elicited the largest effects. FIG. 4B depicts luminescence of the dose-dependent effect of the racemic mixtures of Compounds 6, 9, 11 in SUMO E1 assays. Compounds 6 and 9 showed larger effects than the control compound N106. The potency range for N106 is from 10E-12 M to 10E-8 M.

FIG. 5 discloses the dose-dependent effects of stereoisomers of Compounds 6 on developed force in human cardiac tissue strips that were subjected to maturation protocol (see description below in the section entitled “Maturation of hvCTS”) with maturation media and electrical stimulation of 0.5 Hz from day 7-10, 1.0 Hz from day 10-13, 1.5 Hz from day 13-15, 2.0 Hz from day 16-19, 2.5 Hz from day 19-22 and 3.0 from day 22-24. Contractile data were collected at day 24 post-hvCTS fabrication. Developed forces were measured at 1.5 Hz, 10 minutes after administration of each dose of each stereoisomer. Only hvCTSs with positive force frequency relationships (+FFRs) were tested. Compounds 6-1 and 6-2 (i.e., stereoisomers 1 and 2 of Compound 6 as shown in FIG. 2F) showed positive inotropic effects and/or lusitropic effect effects.

FIG. 6 shows dose-dependent effects of stereoisomers of Compounds 9 on developed force in human cardiac tissue strips that were subjected to maturation protocol with maturation media and electrical stimulation. Contractile data were collected at day 24 post-hvCTS fabrication. Developed forces were measured at 1.5 Hz, 10 minutes after administration of each dose of the stereoisomer. Only hvCTSs with positive force frequency relationships (+FFRs) were tested. Compounds 9-1, 9-2, 9-3 and 9-4 (i.e., stereoisomers 1-4 of Compound 9 as shown in FIG. 2I) showed a decrease in relaxation time that was dose dependent.

FIG. 7 shows designs of a high-throughput Tissue Strip, i.e., TS, platform.

FIG. 8A-E illustrates the fabrication and design of a silicone insert for Design Number 1 and 2 (FIGS. 1A-E). FIG. 8A provides the design of a 4×4 array of inserts (top) and curved posts (bottom), showing side-by-side views of designed curved posts (left) and 3D-printed curved posts (right). The 3D-printed posts were generated using stereolithography (SLA), a process for creating 3D objects in which a computer-controlled moving laser beam is used to build up the required structure, layer by layer, from a liquid polymer that hardens on contact with laser light. FIG. 8B shows the 2-Step casting process comprising using a resin SLA-printed positive casting mold to cast a silicone negative mold (top), which is then used to cast the final positive silicone insert (bottom). FIG. 8C is a top-down view of insert. Dashed boundary line denotes the central tissue seeding area with posts. Solid boundary lines denote areas for electrode insertion. FIG. 8D provides a close-up view of central tissue seeding area and posts. A dashed boundary line denotes boundary of tissue seeding mixture. Channels on the outside of the tissue seeding area lead to the electrode areas to permit electrical stimulation. The channels are designed in such a way that the surface tension of the tissue seeding mixture prevents the liquid from leaking out of the seeding area into the surrounding channels. FIG. 8E is a side-profile view of fabricated plate. Silicone insert (dark gray) is adhered to a 96-well bottomless plate (crosshatch) using a thin layer of silicone-based adhesive (black).

FIG. 9A-C shows deflection of tissue connected to posts. FIG. 9A shows tissue formation attached to the curved posts. The posts are designed with a curved feature intended to retain the tissue at a specific height. As the tissue contracts and exerts force inwards, the post is designed to deflect inward and the distance of deflection is measured.

FIG. 9B provides an example side profile image of tissue compaction around posts. FIG. 9C provides a finite element modeling of a straight post versus a post with curvature. Boundary conditions for the post assumes the post is fixed at the bottom (Y=0) with no x-displacement (ΔX=0). For an applied 10 UN load, the deflection of both posts is similar. Therefore, classical beam bending theory may be applied to derive force of a cardiac tissue from displacement of the top of the curved post. Gray scale represents x-displacement, ranging from 0 μm (black) to 15 μm (light gray).

FIGS. 10A-B provide a high-level schematic view illustrating the versatility of the disclosed bioreactor system and imaging bioreactor platform. FIG. 10A provides a schematic illustration of a bioreactor system comprising an organoid module 10, a computer-controlled detection/recording device 2 (e.g., a camera) for simultaneously imaging up to four organoid cartridges 20 (and optionally saving the images), each containing an organoid 1 (e.g., heart, brain, nerve, liver, kidney, adrenal gland, stomach, pancreas, gall bladder, lung, small intestine, colon, bladder, prostate, uterus, blood, vascular, tumor, eye, or skin), via reflective pyramidal mirror 13. An organoid module 10 may contain a multiple of the same type of organoid 1 or a variety of organoid 1 types. FIG. 10B is a diagram of the imaging bioreactor platform consisting of a computer or data processor 5 controlling an array of organoid modules 10.

FIGS. 11A-C illustrate elements of an embodiment of the bioreactor system that are involved in fluid movements and media flow. FIG. 11A is a schematic of a fluidic exchange system for an organoid cartridge, including fluidic lines, pumps, valves, pressure transducer and fluid tanks. Specific configurations of valves and pumps are used depending on the function, such as FIG. 11B showing aspiration, or FIG. 11C showing fresh feed media addition to the media bath. A detailed description of the illustrated embodiment of the bioreactor system is presented in Example 4.

FIGS. 12A-B present a schematic of fluidic exchange within organoid module 10 to illustrate the creation of a “body-in-a-jar”. FIG. 12A is a graphical representation of fluidic exchange system consisting of fluidic lines, pumps, and valves that direct media between multiple organoid cartridges within a module. A variety of organoid types can be connected to simulate a “body-in-a-jar”. FIG. 12B shows that a heart organoid with sufficient pumping ability could be utilized as the sole biological pump to form a self-powered “body-in-a-jar”. Example 4 provides additional description of these embodiments of the bioreactor system.

FIGS. 13A-B presents methods of flowing fluid into and out of organoids 1. FIG. 13A provides an illustration of an embodiment of the bioreactor system that shows inlet and outlet pathways for media exchange through an organoid 1 controlled by valves (left pane: heart organoid; right pane: liver organoid). FIG. 13B provides a schematic of mechanical stimulation system where a reversible fluidic pump is connected to an organoid 1 for inflation and deflation. The organoid 1 is subject to stretch based on changes in pressure delivered by the stimulation system and the pliability of the organoid 1.

### DETAILED DESCRIPTION

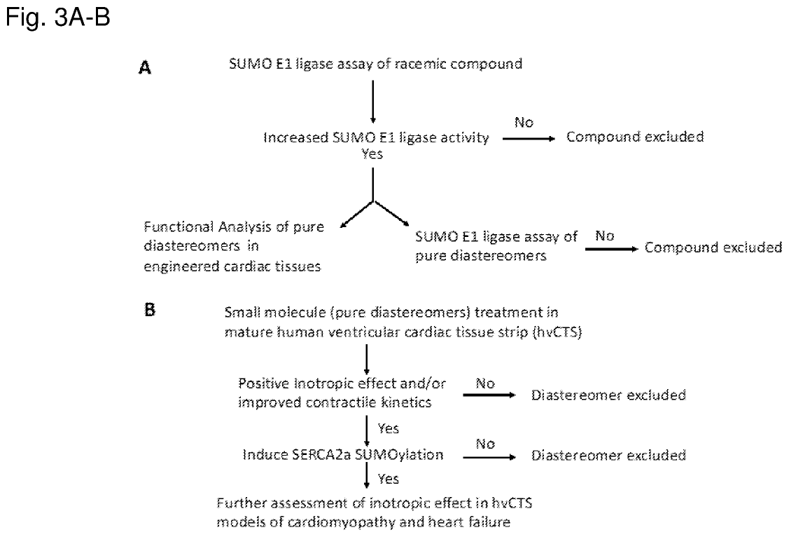

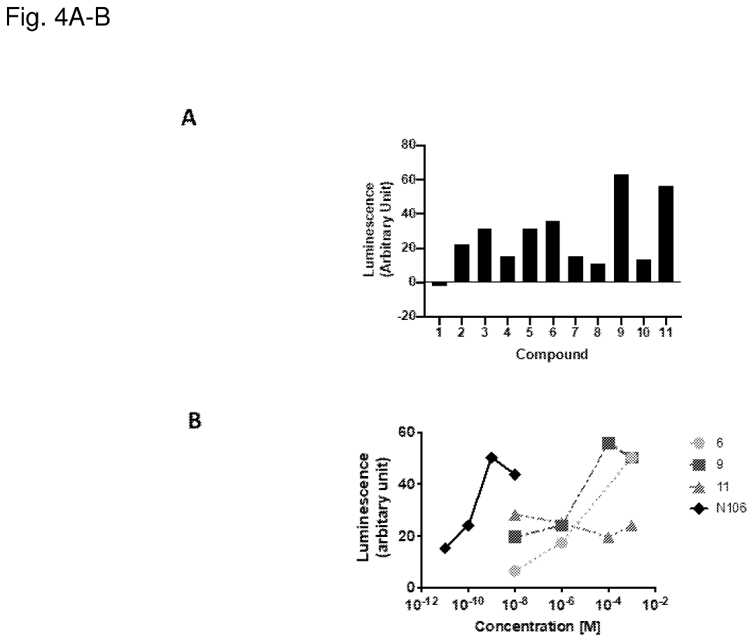

To investigate the effects of carboxamide compounds as SUMOylation activators, the compounds were subjected to an ATP-based assay of SUMO E1 ligase activity. The racemic mixture of each compound was first tested with the SUMO E1 ligase assay to test for SUMO activation activity. Compounds with no SUMO E1 ligase activity were excluded. For compounds with recorded SUMO E1 ligase activity, their four pure stereoisomers were tested for SUMO E1 ligase activity by measuring the adenosine monophosphate (AMP) produced from the E1 ligase reaction as described below. Each pure stereoisomer was also subjected to functional analysis in engineered cardiac tissues (FIG. 3A). The pure stereoisomers of compounds with SUMO E1 ligase activity were administered to human ventricular cardiac tissue strips that had been subjected to a maturation protocol (see below). Contractile parameters, including passive tension, developed force, contraction time, and relaxation time, were measured for positive inotropic effects and/or lusitropic effects attributable to each tested stereoisomer. Those stereoisomers with positive inotropic effects and/or lusitropic effects were tested for their ability to induce SERCA2a SUMOylation using Western blotting as described below. Stereoisomers that could induce SERCA2a SUMOylation were further tested for their inotropic effects, in hvCTS models of cardiomyopathy and heart failure, by fabrication of hvCTS with cardiomyocytes derived from human induced pluripotent stem cells (i.e., hiPSCs) of patients with cardiomyopathy.

### In Vitro SUMO E1 ATP Hydrolysis Assay for SUMO E1 Ligase Activity

Stereoisomers of the test compounds were incubated with SUMO E1 ligase protein and ATP. E1 ligase activation by the test compound was determined by measuring the AMP produced during the enzymatic reaction after removal of the remaining ATP by ATP adenyl cyclase and converting AMP to ADP by polyphosphate-AMP phosphotransferase. The converted ADP was further converted to ATP using adenosine kinase and detected through luciferase reaction.

Measurement of Metrics of hvCTS Cardiac Muscle Function

The effects of compounds on cardiac muscle function as evaluated by the Novoheart hvCTS assay are derived from tracings of twitch force versus time. The twitch force may be calculated from measurements of end-post deflection based on a published beam-bending equation as previously described.^{29, 30} The twitch force tracings are then analyzed from each “heartbeat” of the tissue, and averaged over multiple beats to extract metrics related to the beat rate frequency and beat rate variability, the passive and active tissue forces, and the contraction and relaxation kinetics, as detailed previously in the field²⁹⁻³², and briefly described as follows:

“Heartbeat frequency” is obtained, in some aspects, from the inverse of the time between consecutive beats of the tissue (determined by twitch peak detection) or alternatively by Fast Fourier Transform (FFT) analysis of the force tracing to determine the major frequency component of the signal. “Beat rate variability”, in some aspects, is obtained by calculating the statistical variance in the heartbeat frequency obtained from multiple consecutive twitch tracings.

For each beat, the “passive (or diastolic) force” is the minimum value of the twitch force; the “active (or systolic) force” is the maximum value of the twitch force; and the “contractile (or developed) force” is the difference between the systolic and diastolic forces.

“Contractile kinetics”, in some aspects, are analyzed by examining the twitch force tracing during the increase from the diastolic minimum to the systolic maximum portion of the curve, and these can include the duration of rise time from diastolic to systolic force and related metrics (e.g. duration of rise time from 5% maximum to 95% maximum force, and the like), the area under the curve of rise time from minimum to maximum force, the maximum rate of change of force versus time during contraction (max+dF/dt), and other related metrics.

“Relaxation kinetics”, in some aspects, are analyzed by examining the twitch force tracing during return from the systolic maximum to the diastolic minimum portion of the curve, and these can include the duration of relaxation time from systolic to diastolic force and related metrics (e.g. duration of relaxation time from 95% maximum to 5% maximum force, etc.), the area under the curve of relaxation time from maximum to minimum force, the maximum rate of change of force versus time during relaxation (max-dF/dt), the time constant for an exponential curve fit to the relaxation portion of the twitch force tracing (tau), and other related metrics.

In some aspects, when the hvCTS tissue is electrically paced over a physiologic range of frequencies, such as from about 1.0 Hz (60 bpm) to about 3.0 Hz (180 bpm), the above-described metrics of hvCTS cardiac muscle function metrics is measured at each frequency to determine the heartbeat dependence, the behavior of which is indicative of the physiologic state or health of the tissue. In some particular aspects, measurement of the contractile (developed) force as a function of electrical pacing frequency yields the heartbeat dependent contractile force.

Fabrication and Testing of Human Ventricular Cardiac Tissue Strips (hvCTS)

Three-dimensional multicellular hvCTS myocardial tissues were engineered as previously described^{16, 17}, and those descriptions are incorporated herein in relevant part by reference. Briefly, cardio-clusters from day 15 of hPSC cardiac differentiation were dissociated into single cells and allowed to recover in the incubator for three days before hvCTS construction. Each hvCTS consisted of 1.3×10⁶ cardiac cells differentiated from hPSCs and 1.3×10⁵ human foreskin fibroblasts in a 100 μl ice-cold solution of 2 mg/ml collagen I (0.80-0.95 mg/ml Matrigel, 0.6×PBS, 20 mM NaOH, 0.8× Minimum Essential Medium (Sigma-Aldrich), 1.6 mM HEPES, and 0.1×hvCTS maintenance medium (see composition below). A volume of 100 μl of the final cell collagen mixture is then added to each polydimethylsiloxane (PDMS) bioreactor, consisting of a force-sensing cantilever post at each end of a rectangular well, and returned to the incubator to form the hvCTS attached between the two end posts. High-speed video monitoring of the end-post deflections as the hvCTS beats (either spontaneously or due to electrical stimulation) is then used to calculate the twitch force as a function of time, which is analyzed to evaluate the contractility of the tissue and assess the effects of applied treatments.

Maturation of hvCTS

The hvCTS were maintained in DMEM medium supplemented with 10% newborn calf serum (Gibco), with daily half-medium changes for 7 days, and switched to maturation medium consisting of glucose-free Dulbecco's Modified Essential Medium (DMEM) supplemented with 3 mM glucose, 10 mM L-lactate, 5 mg/ml Vitamin B12, 0.82 mM Biotin, 5 mM Creatine monohydrate, 2 mM Taurine, 2 mM L-carnitine, 0.5 mM Ascorbic acid, 1×NEAA, 0.5% (w/v) Albumax, 1×B27 and 1% KOSR. for a further 18 days to induce maturation.

### SERCA2a SUMOylation Assay

Cardiac proteins were extracted from hvCTS and were immunoprecipitated with a SUMO-1 agarose resin overnight at 4° C. The resins were washed three times with cold lysis buffer (50 mM Tris-HCl (pH 8.0), 150 mM NaCl, 0.1% Triton X-100, 10 mM EDTA, complete protease inhibitor (1 tablet per 10 ml, Roche) and protein phosphatase inhibitor cocktail (Sigma). The immunocomplexes were then resolved by SDS-PAGE. Western blot analyses were performed to measure sumoylated SERCA2a in the hvCTS using a non-commercial SERCA2a-specific antibody custom made by 21^{st} Century Biochemicals, Inc. (Marlborough, MA).

Heart Failure with Preserved Ejection Fraction (HFpEF) is a Global Health Problem Lacking Disease-Modifying Therapeutic Options

Heart failure with preserved ejection fraction (HFpEF) is rapidly becoming the most prevalent cause of heart failure,¹⁸ accounting for over 50% of all HF cases worldwide. The fundamental structural and functional characteristics of the HFpEF heart include delayed and incomplete LV relaxation (lusitropy) and reduced LV compliance, with a relatively normal ejection fraction of 50% or greater.¹⁹ Two pathological characteristics markedly prevalent in patients with HFpEF are cardiac myocyte hypertrophy and cardiac fibrosis, as confirmed in a large, prospective analysis of HFpEF myocardial tissue.^{20,21} Endothelin-1 (ET-1) is a potent stimulator of cardiac myocyte hypertrophy, and is elevated in patients with HFpEF.²² Cardiac fibrosis (CF) is also highly associated with heart failure, including HFpEF, where it has been proposed to play a key role in the progression of the disease,²³ and CF has been associated with mortality in patients with HFpEF.²⁴ In particular, members of the Transforming Growth Factor-β family (e.g., TGF-β1), which are secreted in the cardiac interstitium, influence the activation of specific aspects of the fibrotic response,^{21, 25-28} impacting the synthesis, processing and metabolism of the extracellular matrix.

hvCTS Model of Heart Failure with Preserved Ejection Fraction (HFpEF)

To create an in vitro model of HFpEF, hvCTS myocardial tissue strips are engineered as described herein above for the fabrication of hvCTS, and cultured in DMEM supplemented with 10% NCS, with daily half-medium changes, for five days to allow tissue formation and compaction. To induce the HFpEF phenotype, hvCTS are then treated with TGF-β1 (1 ng/ml) for 4.5 days, followed by treatment with TGF-β1 (1 ng/ml) plus ET-1 (100 nM) for 1 additional day, prior to evaluation of contractile function as described herein above. This combined TGF-β1/ET-1 treatment causes a significant increase in hvCTS passive force and diastolic stiffness and slowing of contraction and relaxation kinetics, with no significant change in systolic or developed force, compared to untreated hvCTS controls, recapitulating some of the key phenotypic features of patients with HFpEF. Transcriptomic analysis of bulk RNAseq data from HFpEF-hvCTS showed patterns of differential gene expression similar to data from HFpEF patient heart tissue, and revealed down-regulation of the SERCA2a gene (ATP2A2) relative to their respective controls, thereby identifying restoration of SERCA2a as a target candidate for mitigating the HFpEF disease traits.

### Testing Efficacy of SUMOylation Activators and E1 Ligase Activators for Treating Human Heart Failure In Vitro

To investigate the efficacy of carboxamide compounds as SUMOylation activators/E1 Ligase activators for treating heart failure in a human-specific in vitro model system, the compounds are used to treat Novoheart's unique in vitro model of human HFpEF. The resulting effects on cardiac functional properties are compared to time-matched untreated HFpEF-hvCTS as negative controls, and compared to time-matched healthy hvCTS as positive controls. Based on the demonstrated mechanism of action of carboxamide compounds and their stereoisomers on cardiomyocyte contractility by activation of SERCA2a and restoring Ca cycling, treatment of HFpEF-hvCTS with SUMOylation activators and E1 Ligase activators reduces diastolic stiffness and hasten twitch force contraction and relaxation kinetics, compared to untreated HFpEF-hvCTS controls, returning the metrics of cardiac function back toward healthy hvCTS values and thereby rescuing key aspects of the HFpEF disease phenotype. Thus, SUMOylation activators/E1 Ligase activators enhance lusitropy in the setting of HFpEF.

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this disclosure is not limited to the particular methodology, protocols, material, reagents, and substances, etc., described herein and as such can vary. The terminology used herein is for the purpose of describing particular embodiments only, and is not intended to limit the scope of the subject matter of the disclosure, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

A series of carboxamide compounds were synthesized to investigate their effect on SUMO activation. The series of carboxamide compounds are derivatives of a base compound having the formula

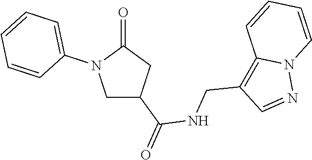 [SMILES: O=C(NCc1cnn2ccccc12)C1CC(=O)N(c2ccccc2)C1]

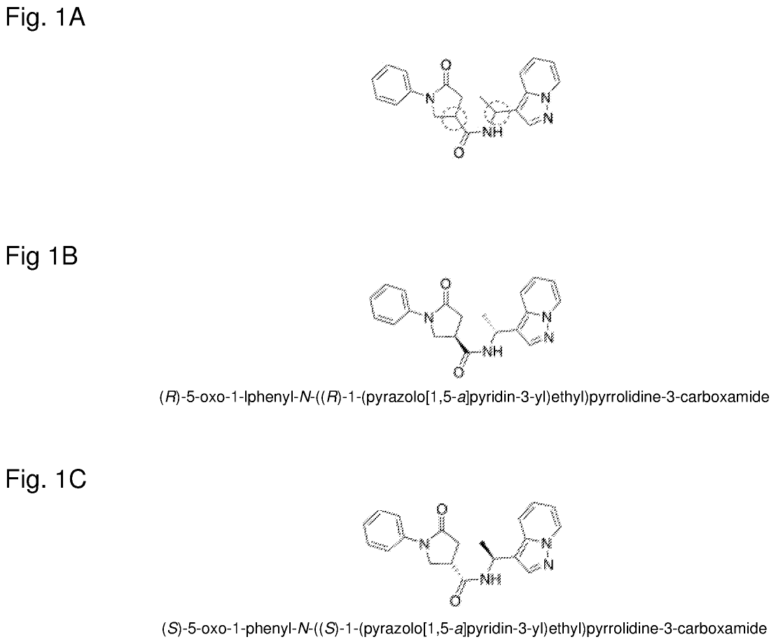

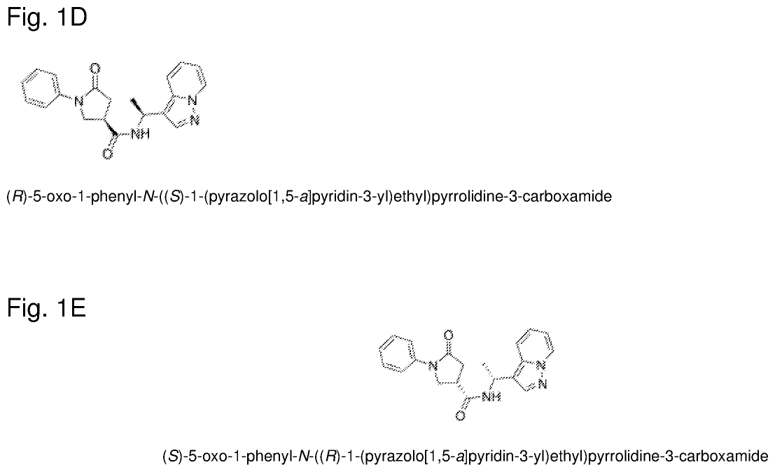

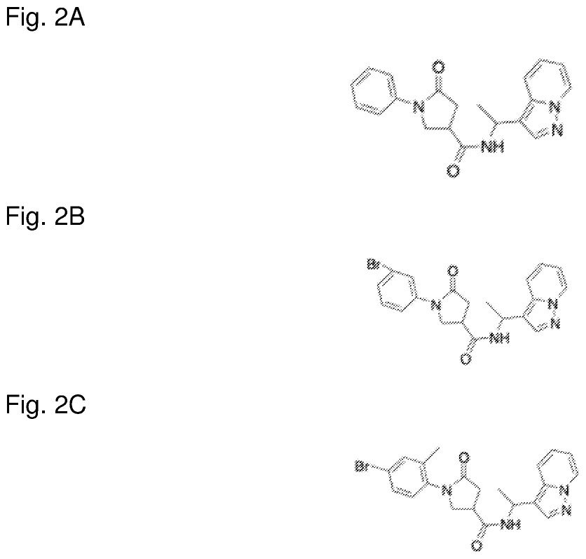

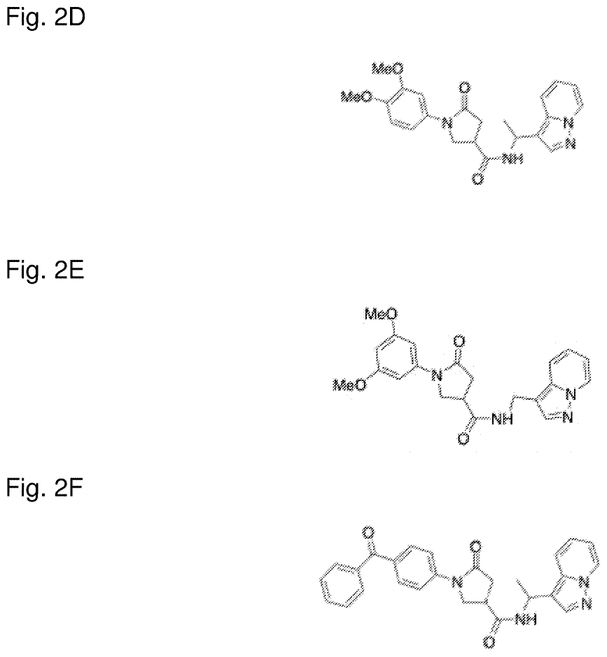

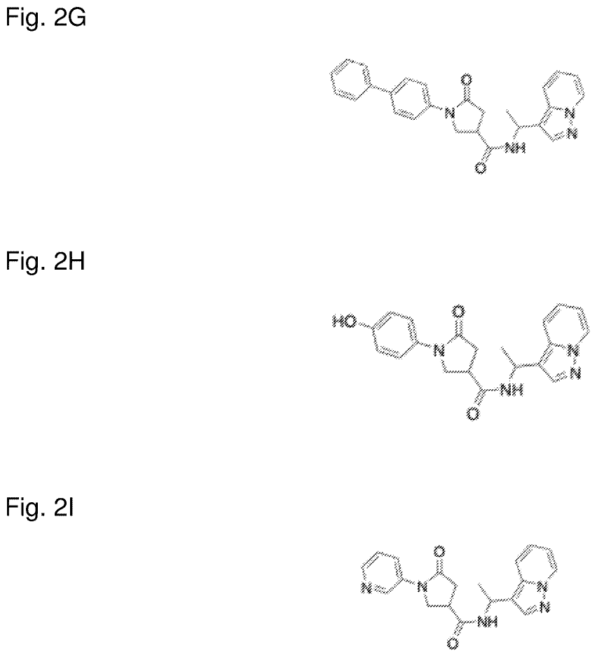

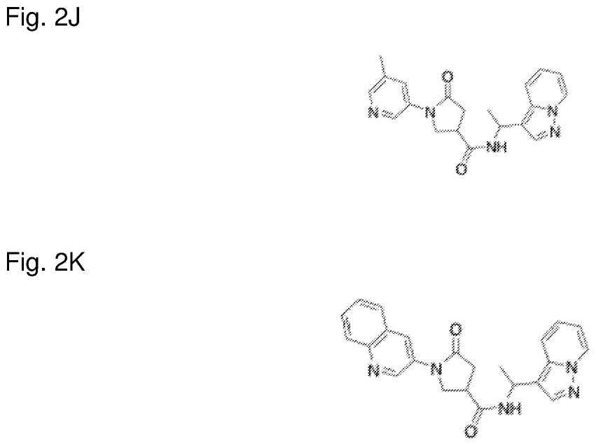

bearing the IUPAC name 5-oxo-1-phenyl-N-(1-(pyrazolo[1,5-a]pyridin-3-yl)ethyl) pyrrolidone-3-carboxamide. The base compound is shown in FIG. 1A, with various substituents attached to the phenyl group in the 1 position, as shown in FIG. 2A-K. These derivatives of the base compound are the series of carboxamide compounds according to the disclosure. Table 1 (set out below) identifies each of the carboxamide derivatives using IUPAC nomenclature, and each of the four stereoisomers of each carboxamide derivative. Each compound has two chiral centers resulting in 4 stereoisomers. Pure stereoisomers were isolated by resolution with HPLC for some assays.

TABLE 1
Carboxamide Derivatives and Stereoisomers

| Exemplary | | | | | |
|---|---|---|---|---|---|
| Compound | IUPAC name | R R stereoisomer | S S stereoisomer | R S stereoisomer | S R stereoisomer |
| Compound 1 | 5-oxo-1-phenyl- | 1-1 | 1-2 | 1-3 | 1-4 |
| (FIG. 2A) | N-(1- | ((R)-5-oxo-1- | ((S)-5-oxo-1- | ((R)-5-oxo-1-phenyl- | ((S)-5-oxo-1- |
| | (pyrazolo[1,5- | phenyl-N-(R)-(1- | phenyl-N-(S)-(1- | N-(S)-(1- | phenyl-N-(R)-(1- |
| | a]pyridin-3- | (pyrazolo[1,5- | (pyrazolo[1,5- | (pyrazolo[1,5- | (pyrazolo[1,5- |
| | yl)ethyl)pyrrolidine- | a]pyridin-3- | a]pyridin-3- | a]pyridin-3- | a]pyridin-3- |
| | 3- | yl)ethyl)pyrrolidine- | yl)ethyl)pyrrolidine- | yl)ethyl)pyrrolidine-3- | yl)ethyl)pyrrolidine- |
| | carboxamide | 3-carboxamide) | 3-carboxamide) | carboxamide) | 3-carboxamide) |
| Compound 2 | 1-(3- | 2-1 | 2-2 | 2-3 | 2-4 |
| (FIG. 2B) | bromophenyl)- | ((R)-1-(3- | ((S)-1-(3- | ((R)-1-(3- | ((S)-1-(3- |
| | 5-oxo-N-(1- | bromophenyl)-(R)- | bromophenyl)-(S)- | bromophenyl)-(S)-5- | bromophenyl)-(R)- |
| | (pyrazolo[1,5- | 5-oxo-N-(1- | 5-oxo-N-(1- | oxo-N-(1- | 5-oxo-N-(1- |
| | a]pyridin-3- | (pyrazolo[1,5- | (pyrazolo[1,5- | (pyrazolo[1,5- | (pyrazolo[1,5- |
| | yl)ethyl)pyrrolidine- | a]pyridin-3- | a]pyridin-3- | a]pyridin-3- | a]pyridin-3- |
| | 3- | yl)ethyl)pyrrolidine- | yl)ethyl)pyrrolidine- | yl)ethyl)pyrrolidine-3- | yl)ethyl)pyrrolidine- |
| | carboxamide | 3-carboxamide) | 3-carboxamide) | carboxamide) | 3-carboxamide) |
| Compound 3 | 1-(4-bromo-2- | 3-1 | 3-2 | 3-3 | 3-4 |
| (FIG. 2C) | methylphenyl)- | ((R)-1-(4-bromo-2- | ((S)-1-(4-bromo-2- | ((R)-1-(4-bromo-2- | ((S)-1-(4-bromo-2- |
| | 5-oxo-N-(1- | methylphenyl)-(R)- | methylphenyl)- | methylphenyl)-(S)-5- | methylphenyl)-(R)- |
| | (pyrazolo[1,5- | 5-oxo-N-(1- | (S)-5-oxo-N-(1- | oxo-N-(1- | 5-oxo-N-(1- |
| | a]pyridin-3- | (pyrazolo[1,5- | (pyrazolo[1,5- | (pyrazolo[1,5- | (pyrazolo[1,5- |
| | yl)ethyl)pyrrolidine- | a]pyridin-3- | a]pyridin-3- | a]pyridin-3- | a]pyridin-3- |
| | 3- | yl)ethyl)pyrrolidine- | yl)ethyl)pyrrolidine- | yl)ethyl)pyrrolidine-3- | yl)ethyl)pyrrolidine- |
| | carboxamide | 3-carboxamide) | 3-carboxamide) | carboxamide) | 3-carboxamide) |
| Compound 4 | 1-(3,4- | 4-1 | 4-2 | 4-3 | 4-4 |
| (FIG. 2D) | dimethoxyphenyl)- | ((R)-1-(3,4- | ((S)-1-(3,4- | ((R)-1-(3,4- | ((S)-1-(3,4- |
| | 5-oxo-N-(1- | dimethoxyphenyl)- | dimethoxyphenyl)- | dimethoxyphenyl)- | dimethoxyphenyl)- |
| | (pyrazolo[1,5- | (R)-5-oxo-N-(1- | (S)-5-oxo-N-(1- | (S)-5-oxo-N-(1- | (R)-5-oxo-N-(1- |
| | a]pyridin-3- | (pyrazolo[1,5- | (pyrazolo[1,5- | (pyrazolo[1,5- | (pyrazolo[1,5- |
| | yl)ethyl)pyrrolidine- | a]pyridin-3- | a]pyridin-3- | a]pyridin-3- | a]pyridin-3- |
| | 3- | yl)ethyl)pyrrolidine- | yl)ethyl)pyrrolidine- | yl)ethyl)pyrrolidine-3- | yl)ethyl)pyrrolidine- |
| | carboxamide | 3-carboxamide) | 3-carboxamide) | carboxamide) | 3-carboxamide) |
| Compound 5 | 1-(3,5- | 5-1 | 5-2 | 5-3 | 5-4 |
| (FIG. 2E) | dimethoxyphenyl)- | ((R)-1-(3,5- | ((S)-1-(3,5- | ((R)-1-(3,5- | ((S)-1-(3,5- |
| | 5-oxo-N-(1- | dimethoxyphenyl)- | dimethoxyphenyl)- | dimethoxyphenyl)- | dimethoxyphenyl)- |
| | (pyrazolo[1,5- | (R)-5-oxo-N-(1- | (S)-5-oxo-N-(1- | (S)-5-oxo-N-(1- | (R)-5-oxo-N-(1- |
| | a]pyridin-3- | (pyrazolo[1,5- | (pyrazolo[1,5- | (pyrazolo[1,5- | (pyrazolo[1,5- |
| | ylmethyl)pyrrolidine- | a]pyridin-3- | a]pyridin-3- | a]pyridin-3- | a]pyridin-3- |
| | 3- | ylmethyl)pyrrolidine- | ylmethyl)pyrrolidine- | ylmethyl)pyrrolidine- | ylmethyl)pyrrolidine- |
| | carboxamide | 3-carboxamide) | 3-carboxamide) | 3-carboxamide) | 3-carboxamide) |
| Compound 6 | 1-(4- | 6-1 | 6-2 | 6-3 | 6-4 |
| (FIG. 2F) | benzoylphenyl)- | ((R)-1-(4- | (S)-1-(4- | ((R)-1-(4- | ((S)-1-(4- |
| | 5-oxo-N-(1- | benzoylphenyl)- | benzoylphenyl)- | benzoylphenyl)-(S)- | benzoylphenyl)- |
| | (pyrazolo[1,5- | (R)-5-oxo-N-(1- | (S)-5-oxo-N-(1- | 5-oxo-N-(1- | (R)-5-oxo-N-(1- |
| | a]pyridin-3- | (pyrazolo[1,5- | (pyrazolo[1,5- | (pyrazolo[1,5- | (pyrazolo[1,5- |
| | yl)ethyl)pyrrolidine- | a]pyridin-3- | a]pyridin-3- | a]pyridin-3- | a]pyridin-3- |
| | 3- | yl)ethyl)pyrrolidine- | yl)ethyl)pyrrolidine- | yl)ethyl)pyrrolidine-3- | yl)ethyl)pyrrolidine- |
| | carboxamide | 3-carboxamide) | 3-carboxamide) | carboxamide) | 3-carboxamide) |
| Compound 7 | 1-([1,1′- | 7-1 | 7-2 | 7-3 | 7-4 |
| (FIG. 2G) | biphenyl]-4-yl))- | ((R)-1-([1,1′- | ((S)-1-([1,1′- | ((R)-1-([1,1′- | ((S)-1-([1,1′- |
| | 5-oxo-N-(1- | biphenyl]-4-yl))-(R)- | biphenyl]-4-yl))- | biphenyl]-4-yl))-(S)- | biphenyl]-4-yl))- |
| | (pyrazolo[1,5- | 5-oxo-N-(1- | (S)-5-oxo-N-(1- | 5-oxo-N-(1- | (R)-5-oxo-N-(1- |
| | a]pyridin-3- | (pyrazolo[1,5- | (pyrazolo[1,5- | (pyrazolo[1,5- | (pyrazolo[1,5- |
| | yl)ethyl)pyrrolidine- | a]pyridin-3- | a]pyridin-3- | a]pyridin-3- | a]pyridin-3- |
| | 3- | yl)ethyl)pyrrolidine- | yl)ethyl)pyrrolidine- | yl)ethyl)pyrrolidine-3- | yl)ethyl)pyrrolidine- |
| | carboxamide | 3-carboxamide) | 3-carboxamide) | carboxamide) | 3-carboxamide) |
| Compound 8 | 1-(4- | 8-1 | 8-2 | 8-3 | 8-4 |
| (FIG. 2H) | hydroxyphenyl)- | ((R)-1-(4- | ((S)-1-(4- | (R)-1-(4- | ((S)-1-(4- |
| | 5-oxo-N-(1- | hydroxyphenyl)- | hydroxyphenyl)- | hydroxyphenyl)-(S)- | hydroxyphenyl)- |
| | (pyrazolo[1,5- | (R)-5-oxo-N-(1- | (S)-5-oxo-N-(1- | 5-oxo-N-(1- | (R)-5-oxo-N-(1- |
| | a]pyridin-3- | (pyrazolo[1,5- | (pyrazolo[1,5- | (pyrazolo[1,5- | (pyrazolo[1,5- |
| | yl)ethyl)pyrrolidine- | a]pyridin-3- | a]pyridin-3- | a]pyridin-3- | a]pyridin-3- |
| | 3- | yl)ethyl)pyrrolidine- | yl)ethyl)pyrrolidine- | yl)ethyl)pyrrolidine-3- | yl)ethyl)pyrrolidine- |
| | carboxamide | 3-carboxamide) | 3-carboxamide) | carboxamide) | 3-carboxamide) |
| Compound 9 | 5-oxo-N-(1- | 9-1 | 9-2 | 9-3 | 9-4 |
| (FIG. 2I) | (pyrazolo[1,5- | ((R)-5-oxo-N-(1- | ((S)-5-oxo-N-(1- | ((R)-5-oxo-N-(1- | ((S)-5-oxo-N-(1- |
| | a]pyridin-3- | (pyrazolo[1,5- | (pyrazolo[1,5- | (pyrazolo[1,5- | (pyrazolo[1,5- |
| | yl)ethyl)-1- | a]pyridin-3- | a]pyridin-3- | a]pyridin-3-yl)ethyl)- | a]pyridin-3- |
| | (pyridin-3- | yl)ethyl)-(R)-1- | yl)ethyl)-(S)-1- | (S)-1-(pyridin-3- | yl)ethyl)-(R)-1- |
| | yl)pyrrolidine-3- | (pyridin-3- | (pyridin-3- | yl)pyrrolidine-3- | (pyridin-3- |
| | carboxamide | yl)pyrrolidine-3- | yl)pyrrolidine-3- | carboxamide) | yl)pyrrolidine-3- |
| | | carboxamide) | carboxamide) | | carboxamide) |
| Compound 10 | 1-(5- | 10-1 | 10-2 | 10-3 | 10-4 |
| (FIG. 2J) | methylpyridin- | (1-(5-methylpyridin- | (1-(5- | (1-(5-methylpyridin- | (1-(5- |
| | 3-yl)-5-oxo-N- | 3-yl)-5-oxo-N-(1- | methylpyridin-3- | 3-yl)-5-oxo-N-(1- | methylpyridin-3- |
| | (1-(pyrazolo[1,5- | (pyrazolo[1,5- | yl)-5-oxo-N-(1- | (pyrazolo[1,5- | yl)-5-oxo-N-(1- |
| | a]pyridin-3- | a]pyridin-3- | (pyrazolo[1,5- | a]pyridin-3- | (pyrazolo[1,5- |
| | yl)ethyl)pyrrolidine- | yl)ethyl)pyrrolidine- | a]pyridin-3- | yl)ethyl)pyrrolidine-3- | a]pyridin-3- |
| | 3-carboxamide | 3-carboxamide) | yl)ethyl)pyrrolidine- | carboxamide) | yl)ethyl)pyrrolidine- |
| | | | 3-carboxamide) | | 3-carboxamide) |
| Compound 11 | 5-oxo-N-(1- | 11-1 | 11-2 | 11-3 | 11-4 |
| (FIG. 2K) | (pyrazolo[1,5- | (5-oxo-N-(1- | (5-oxo-N-(1- | (5-oxo-N-(1- | (5-oxo-N-(1- |
| | a]pyridin-3- | (pyrazolo[1,5- | (pyrazolo[1,5- | (pyrazolo[1,5- | (pyrazolo[1,5- |
| | yl)ethyl)-1- | a]pyridin-3- | a]pyridin-3- | a]pyridin-3-yl)ethyl)- | a]pyridin-3- |
| | (quinolin-3- | yl)ethyl)-1- | yl)ethyl)-1- | 1-(quinolin-3- | yl)ethyl)-1- |
| | yl)pyrrolidine-3- | (quinolin-3- | (quinolin-3- | yl)pyrrolidine-3- | (quinolin-3- |
| | carboxamide | yl)pyrrolidine-3- | yl)pyrrolidine-3- | carboxamide) | yl)pyrrolidine-3- |
| | | carboxamide) | carboxamide) | | carboxamide) |

Racemic mixtures of the 11 exemplary derivatives of carboxamide shown in FIGS. 2A-K were tested for SUMO E1 activity. Of the 11 compounds, Compounds 6, 9 and 11 showed the highest SUMO E1 activity at 1 μM concentration. A dose-dependent increase in SUMO E1 activity could be detected from 0.01 μM to 100 UM for Compounds 6 and 9. These two compounds were chosen for further functional analyses of their stereoisomers.

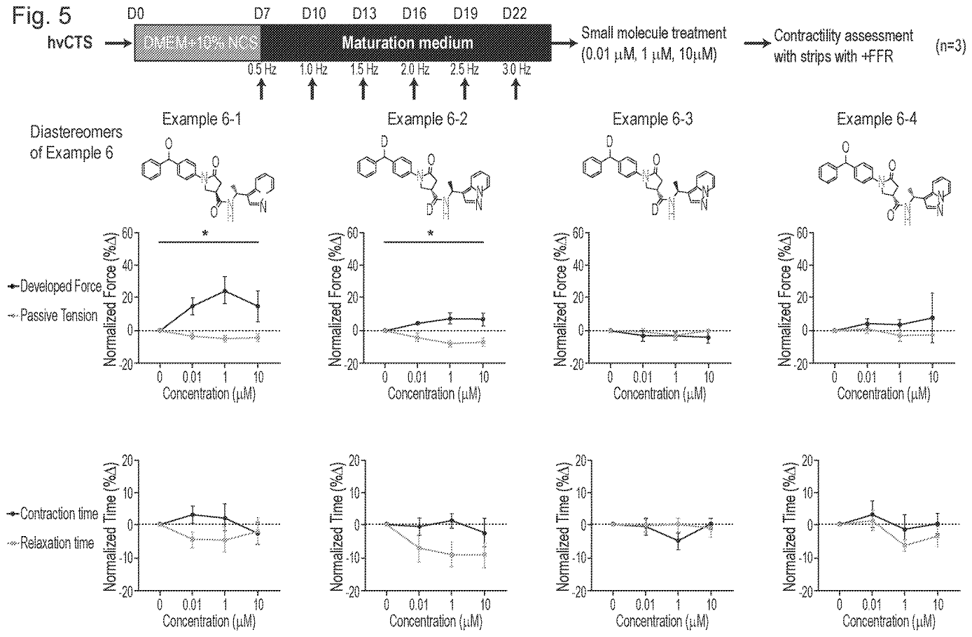

All four stereoisomers of each of Compounds 6 and 9 were tested for their effect on contractility in the engineered cardiac tissue hvCTS that had undergone a maturation protocol to promote maturation of the tissue. For Compound 6, stereoisomer 1 and 2 (Compounds 6-1 and 6-2, respectively, referred to as Example 6-1 and Example 6-2, respectively, in FIG. 5) caused a concentration-dependent increase in developed force, which peaked at 1 μM, while passive tension and contractile kinetics including contraction time and relaxation time did not show any significant difference after treatment with the stereoisomers.

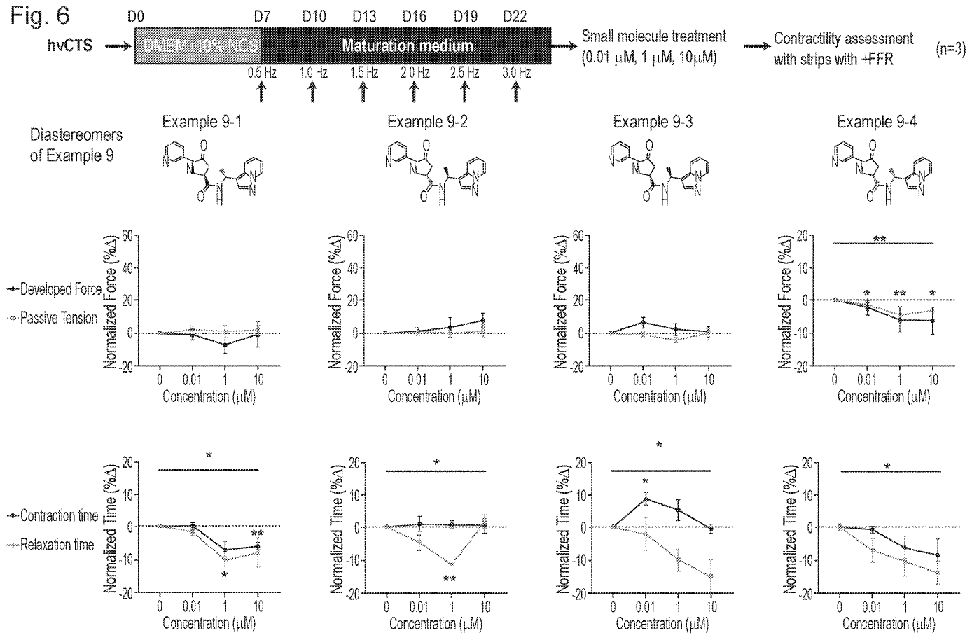

For Compound 9, stereoisomer 4 (Compound 9-4, referred to as Example 9-4 in FIG. 6) caused a small but significant dose-dependent decrease in developed force with a maximum of 10% decrease at 1 μM. All four stereoisomers caused a dose-dependent decrease in relaxation time with the biggest decrease of 15% in stereoisomer 3 at 10 μM.

From the above results, it was determined that stereoisomers 1 and 2 of Compound 6 (Examples 6-1, 6-2 in FIG. 5) and stereoisomer 3 of Compound 9 (Example 9-3 in FIG. 6) of Compound 9 improved contractility and contractile kinetics, respectively.

### Example 2

Provided herein is an exemplary protocol for generating hvCTSs and exemplary devices for use with the hvCTSs in performing the methods according to the disclosure, such as methods of assessing the effects of a compound on cardiac contractility and lusitropy or assessing the cardiotoxicity of a compound. In the protocol for generating hvCTSs, all cell manipulations are performed under aseptic conditions using a HEPA-filtered class II biological safety cabinet and all solutions are sterilized by filtration through a 0.2 μm filter. Tissue construction and function testing are performed under either the same aseptic conditions or in a laminar flow hood.

Initially, H7 hESCs are seeded in preparation for cardiac differentiation. On day 1, the basement membrane matrix is prepared by thawing 150 μl aliquot of hESC-qualified basement membrane matrix on ice overnight at 4° C.

On days 0-4, hESCs are plated on coated plates by diluting the thawed matrix into 12 ml of ice-cold DMEM/F12 and mixed well. One ml of the DMEM/F12-matrix solution is then transferred into each well of a 6-well tissue culture-treated dish. Each aliquot of matrix can coat two 6-well plates. The coated plates are then incubated at room temperature for at least 1 hour. Coated dishes sealed with paraffin can be stored in matrix solution at 4° C. for up to 10 days before use. At approximately 75% confluence, the H7 hESCs are dissociated from the 10 cm dish using 6 ml of non-enzymatic dissociation reagent. After 5 minutes, the cells are gently scraped from the culture surface using a disposable cell scraper and the cell suspension is transferred to a sterile 15 ml centrifuge tube. 0.5 ml of the dissociation solution with the cells is then removed from the 15 ml tube and transferred to a new sterile 15 ml tube (leaving 5.5 ml of the dissociation reagent to further dissociate the hESCs) for stem cell line propagation. The 0.5 ml aliquot of cells is then pelleted at 300×g for 5 minutes at 20° C. The supernatant is then removed and the cell pellet is gently resuspended in 8 ml of pluripotent stem cell media containing 1% penicillin-streptomycin, then transferred to a new, coated, 10 cm tissue culture dish and maintained at 37° C. in 5% CO₂ to maintain the stem cell line. Throughout the protocol, pluripotent stem cell media is maintained on ice during media changes. Meanwhile, 5.5 μl of 10 mM ROCK inhibitor Y-27632 is added to the remaining 5.5 ml of dissociation solution and incubation at room temperature is continued for another 5-10 minutes. The cells are then gently mixed with a 5 ml serological pipette, and incubation is continued until a single cell suspension is achieved. The single cell suspension is then centrifuged at 300×g for 5 minutes at room temperature to pellet the cells. The cells are then resuspended in 5 ml of pluripotent stem cell media and perform a cell count is performed using a hemocytometer. Each well of a 6-well dish is then seeded at a density of 140,000 cells per well. Two 6-well plates are seeded to create a total of six defined tissues, and any remaining cells are disposed. Each well is then filled to 1 ml with pluripotent stem cell media and incubated at 37° C., 5% CO². Twenty-four hours later, media is removed and two ml of fresh pluripotent stem cell media is added to each well. Cell confluence on the plates is checked each day and the differentiation protocol is initiated once the cells reach approximately 75% confluence.

Cells are subjected to the differentiation protocol over days 4-24 of culture. The protocol induces differentiation of human embryonic stem cells to cardiomyocytes. Days 4-7 of culture (days 0-3 of differentiation) result in mesoderm induction. Initially, RPMI differentiation media I is prepared by combining 500 ml RPMI 1640 medium with 10 ml B27 supplement (without insulin) and 5 ml of penicillin-streptomycin stock solution (10,000 IU/ml penicillin; 10,000 μg/ml streptomycin). Aliquot, on ice, into 50 ml tubes and store at 4° C. On culture day 4 (differentiation day 0), mesoderm induction media is prepared by adding 2.4 μl of the small molecule GSK3 inhibitor CHIR99021 (10 mM stock, 6 UM final concentration) to 12 ml of RPMI differentiation media I. Pluripotent stem cell media is then removed from each well and replaced with 2 ml of the mesoderm induction media per well, and the plates are returned to the incubator. Significant cell death typically occurs with the addition of CHIR99021. The monolayer will recover, but it is important to rinse the dead cells away with a DMEM/F12 rinse. On culture day 5 (differentiation day 1), fresh mesoderm induction media is prepared as described above. Spent media is removed from each well and the wells are rinsed once with 1 ml of DMEM/F12 per well. Two ml of fresh mesoderm induction media is then added to each well. Rinsing each day from day 0-10 greatly increases the yield and purity of the cardiac myocytes. On day 6 (differentiation day 2), the cardiac induction media is removed and the wells are rinsed once with 1 ml DMEM/F12 per well. The rinse is replaced with 2 ml RPMI differentiation media I (no small molecules added but still with the B27 (without insulin) supplement), and the cells are returned to the incubator.

On days 7-13 (differentiation days 3-10), cardiac mesoderm is induced using the following protocol. On day 7 (differentiation day 3), cardiac mesoderm induction media is prepared by adding 6 μl of the small molecule Wnt inhibitor IWR-1 (10 mM stock, 5 M final) to 12 ml of RPMI differentiation media I. Media is removed from each well, the well is rinsed once with 1 ml DMEM/F12 per well, and the rinse is replaced with 2 ml of cardiac mesoderm induction media per well. On day 8 (differentiation day 4), an additional 12 ml of cardiac mesoderm induction media is prepared as described above. Media added the previous day is removed, the wells are rinsed once with 1 ml DMEM/F12 per well, and the rinse is replaced with 2 ml of fresh cardiac mesoderm differentiation media per well, with the cells being returned to the incubator. On each of days 9-10 (differentiation days 5-6), the cardiac mesoderm induction media is removed and the wells are rinsed with 1 ml of DMEM/F12. Two ml of fresh RPMI differentiation media I (no small molecules added but still with the B27 (without insulin) supplement) are added to each well, and the plates are returned to the incubator.

Days 11-24 (differentiation days 7-20) lead to hES-derived Cardiac Myocyte Organization/Maturation. Initially RPMI differentiation media II is prepared by combining 500 ml RPMI 1640 with 10 ml B27 supplement (with insulin) and 5 ml of penicillin-streptomycin stock solution. The differentiation media is aliquoted, on ice, into 50 ml tubes and stored at 4° C. On day 11 (differentiation day 7), RPMI differentiation media (without insulin) is removed from each well and the wells are rinsed with 1 ml DMEM/F12 per well. Two ml of the new RPMI differentiation media II (with insulin) is added to each well and the cells are returned to the incubator. Spontaneous beating should first be observed between days 7 and 10. If beating is not observed during this time, it typically indicates poor differentiation efficiency. The protocol can be continued until day 15 to observe beating, but if no beating is observed by day 15, it is best to start a new differentiation. On each of days 12-24 (differentiation days 8-20), spent differentiation media is removed and replaced with 2 ml of fresh RPMI differentiation media II per well to permit cell maturation and organization of the beating monolayer. Depending on the residual cell death, it may be necessary to rinse with 1 ml of DMEM/F12 through differentiation day 10.

At day 24 (differentiation day 20), the isolation of cardiac myocytes and fibroblast-like Cells is undertaken. Initially, cells are dissociated from the adherent monolayer of cells in the wells of the incubation plates by removing the differentiation media and rinsing the cells in each well once with 1 ml PBS. The monolayer cells are then dissociated by adding 1 ml of the enzymatic dissociation solution (0.04% Trypsin/0.03% EDTA) to each well. Plates are then moved into the incubator for 10 minutes. Meanwhile, 12 μl of ROCK inhibitor is added to 6 ml of trypsin neutralization solution. One ml of the trypsin neutralization solution containing the ROCK inhibitor is then gently added to each well of the plate to neutralize the trypsin solution. A sterile transfer pipette is then used to gently mix each well to break apart the cell clusters. All 12 ml from a 6-well plate are then added to a 15 ml centrifuge tube. Three ml of PBS are then transferred to one well of the plate, and then sequentially transferred to each subsequent well to collect any residual cells in the dish into the single aliquot of three ml of PBS. The 3 ml of PBS containing cells is then transferred from the final well into the same 15 ml centrifuge tube containing the originally harvested cells, and the cell suspension is centrifuged at 300×g for 5 minutes at 4° C. to pellet the cells.

At this stage of the protocol, cells are prepared for live cell-sorting by FACS. A staining buffer is prepared by adding 5 ml of fetal bovine serum to 45 ml of PBS on ice with 50 μl of ROCK inhibitor. The supernatant is removed from the tube containing the pelleted cells and those cells are resuspended in 1.2 ml of staining buffer. 200 μl of the cell suspension is then transferred to a new, pre-chilled, 50 ml centrifuge tube on ice to provide a negative staining control. The remaining 1 ml of cell suspension is transferred to a new, pre-chilled, 50 ml centrifuge tube on ice and 2 μl SIRPα-PE/Cy7 (1:500 dilution) is added along with 4 μl of CD90-FITC (1:250 dilution). The cell suspension is gently mixed with a transfer pipette and returned to the ice. Both the negative control and the sample are incubated on a rocker shaker at 4° C. for 1 hour. Sample collection tubes were prepared by adding 3 ml of RPMI media (with insulin) to two 15 ml centrifuge tubes. Three μl of ROCK inhibitor was added to each tube and the tubes were stored on ice. Stained cells were centrifuged at 300×g for 5 minutes at 4° C. and the pelleted cells were rinsed twice with at least 10 ml of ice-cold PBS per rinse. One μl of DAPI (1 μg/ml) was then added to 5 ml of staining buffer, and each sample pellet was gently resuspended in 1-3 ml of the DAPI-containing staining buffer using a transfer pipette. 500 μl of staining buffer (with no DAPI added) was added to the negative control. Both the negative control and the sample were then gently filtered through a 40 μm cell strainer to remove clumps of cells, and the strained cell suspensions were transferred to polystyrene FACS tubes on ice. Samples were then immediately subjected to sorting using the cell sorter.

Similar to established live cell sorting methods, the negative control was used to set the gates, select for live cells (DAPI negative), and collect both the FITC+ (i.e., CD90+ fibroblast) and PE/Cy7+ (i.e., SIRPα+ cardiomyocyte) populations independently at 20 psi. After setting the gates, the negative control can be fixed in 4% PFA to determine differentiation efficiency by staining for cardiac troponin-T. Also contemplated is the use of an isotype control rather than an unstained control to set the gates in order to compensate for non-specific antibody binding. A so-called fluorescence minus one (FMO) control is another possibility. Due to the clear bimodal distribution of the FITC, DAPI and PE-Cy7 signals, we gated from the positive population, conservatively aiming far into the positive gate, which possibly excluded some true positives but helped to minimize any false positives.

The following describes cell reaggregation in preparation for tissue engineering. After the cell sort, Cells in the collection tubes are centrifuged and each cell pellet is resuspended in 1 ml of DMEM containing 10% neonatal bovine serum, 1% penicillin-streptomycin, and 0.2% amphotericin B (“NBS media”). The SIRPα+ and CD90+ cells are combined in a 3:1 ratio and plate the combined cells are plated in a non-tissue culture-treated petri dish at a density of 2 million cells per 60 cm² (10 cm dish). Ten ml of NBS media and 10 μl of ROCK inhibitor Y-27632 are then added. The cell suspension is then placed in a tissue culture incubator for 48 hours to allow cell reaggregation into small clusters.

Additional steps are performed to engineer human cardiac tissue. In some embodiments, a multi-tissue bioreactor is fabricated. A PDMS master mold is machined by drilling six evenly spaced holes of 0.5 mm diameter into a 9×33×3.25 mm cuboid of polytetrafluoroethylene. Using an end mill, a frame is machined from polysulfone that typically measures 25×35×11 mm³. The purpose of the frame is to hold the PDMS posts (constructed from the cast made above) in alignment with the wells in the baseplate. Using a 1 mm end mill, machine 6 wells (6×1×1 mm³) are machined 4 mm apart into a 20×40×5 mm³ piece of black polytetrafluoroethylene to form the baseplate. The elastomeric base and curing agent for polydimethylsiloxane (PDMS) are then mixed in a 10:1 w/w ratio and added to the custom polytetrafluoroethylene mold to create two rows of six force-sensing posts, and the device is incubated overnight and under vacuum at 80° C. After curing, the PDMS is gently removed from the master mold and the top of each post is marked with a black permanent marker for enhanced contrast and automated real-time post deflection tracking. Polytetrafluorethylene is fairly soft and can be damaged easily. Use care when cleaning the master mold prior to PDMS casting to ensure longevity of the system and consistent PDMS post geometry. A 0.5 mm wire, in various aspects, is used to clean the holes for the posts after each use, and care is taken to not scrape the inside of the holes. An alternative is to de-gas the PDMS under vacuum for several hours at room temperature, then let the mixture cure at ambient pressure. This may lead to fewer residual gas bubbles forming in the PDMS during the curing process. All components are sterilized in a steam autoclave. The PDMS master mold and the polysulfone frame are both reusable. The PDMS posts created from the cast are also reusable, but only for approximately 10 uses. Additional PDMS posts can be created as needed using the master mold, however.

The cardiac cells reaggregated as described above are then collected. The reaggregated cells are removed from the incubator and all 10 ml of the reaggregation media is transferred from the tissue culture plate (dish) to a 50 ml centrifuge tube. The plate is then rinsed with 3 ml of PBS and the rinse is transferred to the same 50 ml centrifuge tube containing the reaggregation media. Three ml of 0.04% Trypsin/0.03% EDTA is added to the 10 cm dish and returned to the incubator for 5 minutes. After 5 minutes, the plate is examined using an inverted compound microscope at 10× magnification to ensure complete cell dissociation from the dish. If some residual clusters are still attached, the plate is gently agitated to detach the cell clumps. If the clusters remain attached, the dish is returned to the incubator for another 2-3 minutes. Do not incubate longer than ten minutes or significant cell death may occur. Once all cells are detached, 3 ml of trypsin neutralization solution is added. Gently mix the neutralization solution with the trypsin-cell solution and transfer the mixture to the 50 ml centrifuge tube containing the reaggregation media. The entire dish is then rinsed with 5 ml of PBS and transferred to the same 50 ml tube containing the cells. The cells are then centrifuged at 300×g for 5 minutes at room temperature. The supernatant is removed and the cell pellet is resuspended in 1 ml of NBS media, then transferred to a 1.5 ml microcentrifuge tube. The cells in the microcentrifuge tube are then centrifuged at 300×g for 5 minutes at room temperature and the supernatant is removed. The cardiac cells (CD90+ stromal cells and SIRPα+ myocytes) are now ready for tissue construction.

In some embodiments, supplemental cells of interest are also collected. In addition to the defined tissues containing only SIRPα+ cardiomyocytes and CD90+ fibroblast-like cells, it is possible to add additional cells of interest to interrogate their effect on tissue function. For example rat MSCs have been shown to enhance the function of rat engineered cardiac tissues. The following optional step describes the collection of supplemental cells for the defined system.

Collect the supplemental cell type of interest (e.g., mesenchymal stem cells) using 0.25% trypsin/0.1% EDTA. Pellet the cells at 300×g for 5 minutes at room temperature, then resuspend the cells in 5 ml of appropriate cell culture media for the cell type of interest. For instance, for MSCs, use DMEM supplemented with 20% fetal bovine serum, 1% penicillin-streptomycin and 0.2% amphotericin-B to culture the cells. A cell count is then performed using a hemocytometer, and the cells are again centrifuged at 300×g for 5 minutes at room temperature. The supernatant is removed and the cells are resuspended in 1 ml of cell culture media for transfer to a 1.5 ml microcentrifuge tube. The transferred cells are again centrifuged at 300×g for 5 minutes at room temperature. The supernatant is removed and the supplemental cells are now ready for tissue construction. If the supplemental cells are added at a concentration of 10% of the total cell number in the tissue, then for the defined tissues, this will require 50,000 supplemental cells per tissue as each tissue contains 500,000 cardiac cells (both CD90+ stromal cells and SIRPα+ myocytes).

The materials generated as described above are useful in creating human engineered cardiac tissues, such as human ventricular cardiac tissue strips (hvCTSs). For this procedure, all solutions should be stored on ice and the cells should be maintained at room temperature. All volumes listed below are per tissue. Typically, approximately six tissues can be constructed from two 6-well plates of cardiac differentiations.

Initially, 60.0 μl of the 5 mg/ml collagen stock solution is diluted to 3.125 mg/ml with 1.5 μl of 1 M NaOH, 9.6 μl of 10×PBS and 24.9 μl of sterile ultrapure deionized water. It is important to avoid the introduction of air bubbles to each solution during preparation, as air bubbles will disrupt proper tissue formation. 12.0 μl of both 10×MEM and 0.2 N HEPES pH 9 are added to the dilute collagen mixture to create the collagen mix. Add both solutions down the side of the 15 ml centrifuge tube in order to avoid the introduction of air bubbles into the collagen mix. Add the basement membrane matrix (final concentration of 0.9 mg/ml) to the collagen mix and store on ice to create the final tissue mix. The final concentration of collagen should be 2 mg/ml. 500,000 reaggregated cells are then added to the tissue mix (myocyte+ fibroblast concentration is 20 million/ml) and the mix is brought to a final volume of 25 μl per tissue with either cell-free NBS media or 50,000 cells of the supplemental cell type of interest (e.g., hMSCs) and mixed well to form an even cell suspension. The number of supplemented cells will vary for different applications. In this example, 10% supplementation is used. With care, 25 μl of the cell suspension is pipetted into each of the six wells in the bioreactor baseplate, without introducing air bubbles into the wells. Next, two rows of the PDMS force sensors are pushed into place on either side of the polysulfone frame, forming 6 pairs of opposing posts. The frame is then inverted on top of the baseplate so that one pair of posts enters each well containing cell suspension. The polysulfone frame is constructed to include tabs to aid in alignment of the PDMS. Carefully place the bioreactor, baseplate down, into a 60 mm dish. Place the 60 mm dish without its cover inside of a 10 cm dish, place the 10 cm cover on top of the 10 cm dish, and move the entire bioreactor assembly into the tissue culture incubator, waiting two hours for the tissue to gel. After 2 hours, remove the bioreactor from the incubator and add 14 ml of NBS media to the entire assembly, which is enough to cover the baseplate. Return the bioreactor to the incubator, and change half of the media every day. Forty-eight hours later, carefully remove the baseplate by gently moving each side of the baseplate off the frame a few millimeters at a time, change the media, then return the bioreactor, tissues facing down, to the media. Continue changing half of the NBS culture media every day. Spontaneous contractions of the tissue can be observed as early as 3 days after tissue construction, generating measureable twitch forces as early as day 5 to 7.

### Example 3

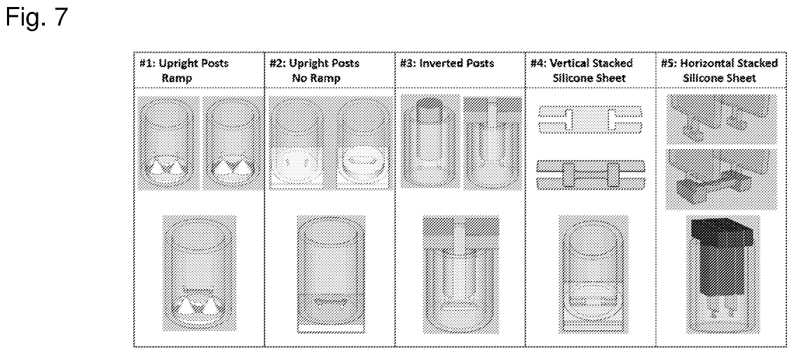

This Example discloses exemplary embodiments of tissue strips, such as human ventricular cardiac tissue strips (hvCTSs), useful in the devices and methods of the disclosure. Additional description of tissue strips is provided in U.S. Pat. Pub. No. 2020/036996, incorporated herein by reference. The tissue strip (TS) platform (e.g., a CTS platform) can be fabricated in multiple different configurations (FIG. 7). Each configuration is designed for the following: (1) 96-well format, (2) two anchor points to create aligned, elongated tissue, (3) one tissue strip per well, (4) in-well optical monitoring of contractile force, (5) in-well electrical pacing compatibility, and (6) design elements for standardization of tissue z-height. In some embodiments, the platform can be scaled to other standard multi-well formats, such as, but not limited to, 48-well, 24-well, 12-well, 6-well formats, and the like.

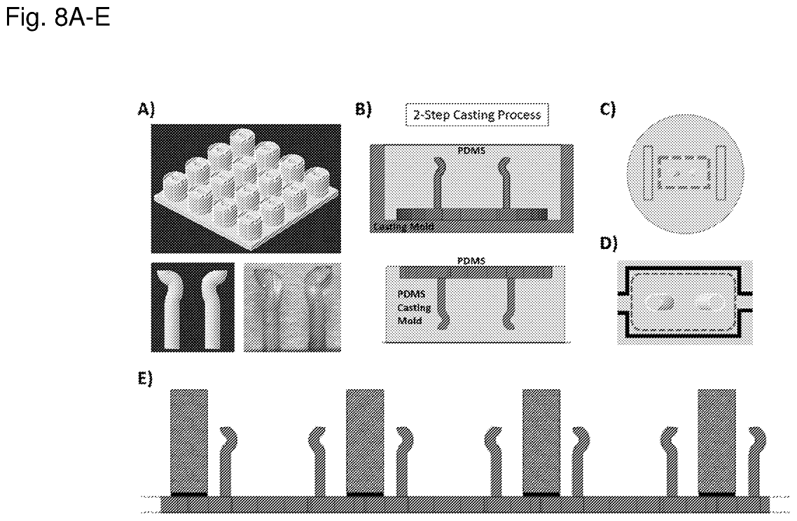

Designs of 96-well inserts with curved upright posts (FIG. 8A) were produced using AutoCAD (Autodesk), and fabricated in a 2-step casting process (FIG. 8B). The design includes a central tissue seeding area connected to two electrode retaining areas that permit electrical field stimulation of the tissue (FIG. 8C). The channels that connect the tissue seeding area to the electrode area are designed such that surface tension of the tissue seeding mixture prevents leakage of the mixture out of the seeding area (FIG. 8D). Positive master molds were 3D-printed (ProJet 6000HD, 3D Systems, or Form 3, Formlabs) using stereolithography (SLA) to create a 4×4 array of inserts. In some embodiments, insert size is increased (e.g., 4×8 or 8×12 array). The master molds were silanized using trichloro (1H, 1H,2H,2H-perfluorooctyl) silane (PFOCTS) before use to prevent adhesion and facilitate separation after casting. Polydimethylsiloxane (PDMS, Sylgard 184, Dow Corning) was mixed at 10:1 (base: curing agent) and degassed to remove trapped air bubbles. In some embodiments, the PDMS mixing ratio of base: curing agent is altered (e.g., 5:1 to 20:1) to adjust material stiffness. Using aluminum foil enclosures, 25 g of PDMS was cast over each master mold and further degassed. The PDMS was cured for 48 hours at room temperature followed by 2 hours at 65° C. The resulting PDMS negative molds were silanized using PFOCTS, then cast with 7 g of PDMS to form positive inserts for the 96-well plate. After degassing, the PDMS was cured for 2 hours at 65° C. The PDMS inserts were sealed to the base of a polystyrene bottomless 96-well plate (Greiner Bio-One) using adhesives (FIG. 8E) such as silicone adhesive (Silastic Medical Adhesive Silicone, Type A), cyanoacrylate (Loctite 435) or medical tape (double-sided 3M Microfluidic Medical Tape).

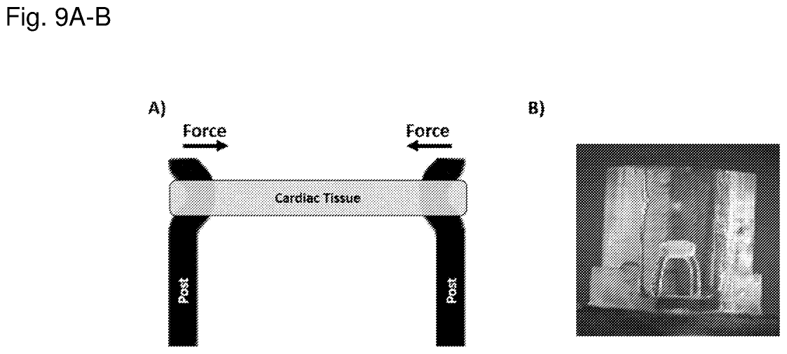

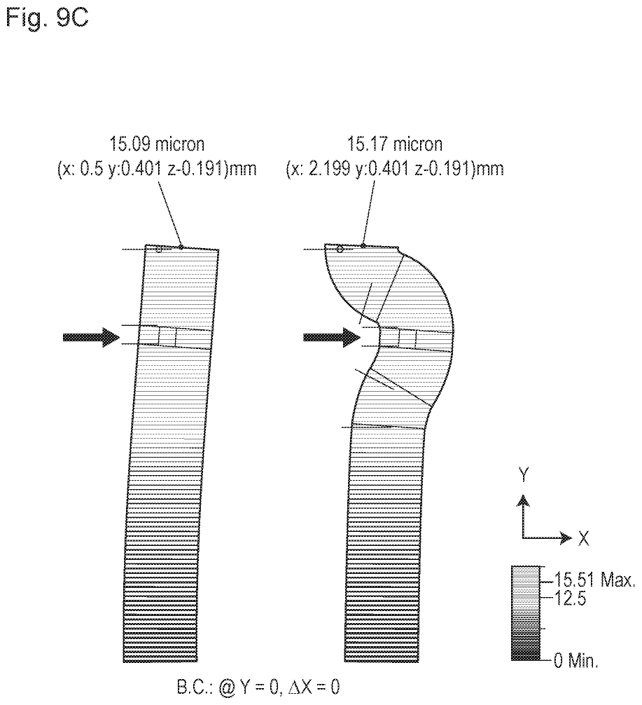

Exemplary tissue strip deflection using a cardiac tissue strip with curved posts is illustrated in FIG. 9A-B. The use of curved posts helps to fix the height of the tissue strip, aiding in the observation of the behavior of the tissue strip in response to various added compounds and/or electrical stimuli.

Consistent with the preceding description, exemplary microfabricated devices according to the disclosure comprise two posts, or anchors, to which a tissue strip attaches. The anchors are flexible to allow movement of the tissue strip, which allows measurement of any change in force exerted by the tissue strip in relation to changes in conditions. The microfabricated devices are amenable to incorporation in high-throughput assay formats. For example, high-throughput assays using the microfabricated devices can be formatted for 96-well clear polystyrene plate formats that are suitable for automated measurements and monitoring using plate readers known in the art. In addition to compatibility with 96-well plate formats, the microfabricated devices can be formatted for 6-well, 12-well, 24-well, and 48-well plates. In some cases, plates are made of opaque material for use in fluorescent assays. Furthermore in addition to measurements, standard plate formatting is important for adaptation to other automated machine systems such as media exchange, reagent addition, and mixing. The microfabricated devices are also compatible with electrical stimulation of tissue strips attached to the anchors. In some embodiments, a trough is provided for seeding that allows electrical stimulation by concentrating seeding in specific location due to surface tension. The microfabricated devices disclosed herein provide an advantage in both force output and electrophysiology over designs characterized by tissue rings. It is therefore desirable to design linear tissues amenable to high-throughput measurement to overcome these shortcomings of tissue rings.

Described herein are various designs of a tissue strip (TS) platform compatible with standard cell culture plates for use in high-throughput applications, including automated robotic multi-well plate handling systems. See, e.g., FIGS. 7-9A-B. In particular, the tissue strip platform is suitable for the development and manipulation of a cardiac tissue strip (CTS), although a variety of tissue strips are contemplated by the disclosure, including visceral organ tissue strips such as liver, stomach, pancreas, gall bladder, kidney, small intestine, colon, urethra, ureter, bladder, prostate, uterus, and ovary tissue strips as well as eye, skin, brain, tongue, esophagus, vascular, tendon, ligament, skeletal muscle and smooth muscle tissue strips.

In some embodiments, the microfabricated device comprises an intact and functional miniature tissue for high-throughput screening of the potential toxicity of compounds, wherein the device comprises at least two biocompatible posts and a substrate to which the posts are attached, wherein the distance separating at least two posts is at least 0.5 mm, and wherein each post comprises an elastomeric material, a force sensor, and a feature for tethering a tissue strip, wherein the tissue strip comprises a composition comprising cells of at least one force-generating cell type and one extracellular matrix type, further wherein the microfabricated device is suitable for monitoring tissue strip position over time, in situ. In some embodiments, the suitability for monitoring tissue strip position over time is further suitable for providing a measurement of tissue strip movement in situ. In some embodiments, the force-generating cell type is a smooth muscle cell type, a skeletal muscle cell type, a cardiac muscle cell type, a fibroblast cell type, a neutrophil cell type, an eosinophil cell type, a basophil cell type, a monocyte cell type, a lymphocyte cell type, a glial cell type, a chondrocyte cell type, an osteoblast cell type, an osteoclast cell type, an osteocyte cell type, a keratinocyte cell type, a melanocyte cell type, a Merkel cell type, a dendritic cell type, an endothelial cell type, an epithelial cell type, a white adipocyte cell type, a brown adipocyte cell type, an esophageal cell type, a pharynx cell type, a larynx cell type, a lung cell type, an hepatocyte cell type, a bladder cell type, a kidney cell type, a stomach cell type, a gallbladder cell type, beta islet cell type, a spleen cell type, a small intestine cell type, or a colon cell type. In some embodiments, the force-generating cell type is a stem cell-derived cell type, such as a myocyte cell type. In some embodiments, the myocyte cell type is a cardiomyocyte cell type, which may be a primate, horse, cow, goat, sheep, zebrafish, pig, dog, rat, mouse, or a human cardiomyocyte cell type, e.g., a human ventricular cardiomyocyte cell type.

In some embodiments, the elastomeric material is silicone, such as polydimethylsiloxane, polyurethane, polyethylene, or polyacrylamide. In some embodiments, the substrate is thin-layer silicone, such as thin-layer polydimethylsiloxane, thin-layer polyurethane, thin-layer polyethylene, or thin-layer polyacrylamide. In some embodiments, the feature for tethering a tissue strip is a curve in the post.

In some embodiments, the microfabricated device further comprises a recording device to monitor tissue strip position or to detect movement of a tissue strip. In some embodiments, the microfabricated device further comprises two unipolar electrodes for field stimulation, a bipolar micro-electrode for point contact stimulation, or a micro-cannula for contacting a tissue strip with an electrical stimulus or a modulator of a biological tissue activity, e.g., a microfabricated device comprising two unipolar electrodes for field stimulation or a bipolar micro-electrode for point contact stimulation.

Another aspect of the disclosure is drawn to a system for measuring tissue activity comprising: (a) a microfabricated device as disclosed herein; and (b) a recording device for capturing the force detected by at least one force sensor of the microfabricated device. In some embodiments, the tissue comprises a cell type that is a smooth muscle cell type, a skeletal muscle cell type, a cardiac muscle cell type, a fibroblast cell type, a neutrophil cell type, an eosinophil cell type, a basophil cell type, a monocyte cell type, a lymphocyte cell type, a glial cell type, a chondrocyte cell type, an osteoblast cell type, an osteoclast cell type, an osteocyte cell type, a keratinocyte cell type, a melanocyte cell type, a Merkel cell type, a dendritic cell type, an endothelial cell type, an epithelial cell type, a white adipocyte cell type, a brown adipocyte cell type, an esophageal cell type, a pharynx cell type, a larynx cell type, a lung cell type, an hepatocyte cell type, a bladder cell type, a kidney cell type, a stomach cell type, a gallbladder cell type, beta islet cell type, a spleen cell type, a small intestine cell type, or a colon cell type. In some embodiments, the tissue comprises a stem cell-derived cell type. In some embodiments, the tissue comprises a myocyte cell type, such as a cardiomyocyte cell type, which may be a primate, horse, cow, goat, sheep, zebrafish, pig, dog, rat, mouse, or a human cardiomyocyte cell type, e.g., a human ventricular cardiomyocyte cell type. In some embodiments, the system further comprises two unipolar electrodes for field stimulation, a bipolar micro-electrode for point contact stimulation, or a micro-cannula for contacting a tissue composition comprising the cells of at least one cell type with an electrical stimulus or a modulator of a biological tissue activity.

Yet another aspect of the disclosure is a method for assaying the toxicity of a test compound on a tissue, such as the tissue in a tissue strip according to the disclosure, comprising: (a) contacting the tissue strip with the test compound; (b) exposing the tissue strip to an electrical stimulus; and (c) measuring the response of the tissue strip, wherein the response of the tissue strip is compared to a control or baseline measurement of a tissue strip of the same cell type or types not exposed to the test compound. In some embodiments, the tissue comprises a cell type that is a smooth muscle cell type, a skeletal muscle cell type, a cardiac muscle cell type, a fibroblast cell type, a neutrophil cell type, an eosinophil cell type, a basophil cell type, a monocyte cell type, a lymphocyte cell type, a glial cell type, a chondrocyte cell type, an osteoblast cell type, an osteoclast cell type, an osteocyte cell type, a keratinocyte cell type, a melanocyte cell type, a Merkel cell type, a dendritic cell type, an endothelial cell type, an epithelial cell type, a white adipocyte cell type, a brown adipocyte cell type, an esophageal cell type, a pharynx cell type, a larynx cell type, a lung cell type, an hepatocyte cell type, a bladder cell type, a kidney cell type, a stomach cell type, a gallbladder cell type, beta islet cell type, a spleen cell type, a small intestine cell type, or a colon cell type. In some embodiments, the tissue comprises a stem cell-derived cell type. In some embodiments, the tissue comprises a myocyte cell type, such as a cardiomyocyte cell type, which may be any mammalian cardiomyocyte cell type, including a primate, horse, cow, goat, sheep, zebrafish, pig, dog, rat, mouse, or a human cardiomyocyte cell type, e.g., a human ventricular cardiomyocyte cell type. In some embodiments of the method, the response is contractile force. In some embodiments, the microfabricated device further comprises a recording device that detects the presence or absence of movement of the tissue strip, wherein movement of the tissue strip results from a change in contractile force.

### Example 4

The disclosure contemplates devices comprising tissue strips for use in assessing the cytotoxicity of compounds, as exemplified in Example 3. The disclosure further contemplates assay systems involving one or more tiers, such as a multi-tiered assay system, in which a first tier tissue strip is used for an initial assessment of the cytotoxicity of compounds, for example in a high-throughput format. Beyond assessing cytotoxicity using devices comprising the tissue strips, the disclosure comprehends an assay system, which in some embodiments is a second tier of an assay system, in which organoid chambers are used to generate organoids that are then used in cytotoxicity assays of compounds. Additional description of organoid chambers and bioreactors comprising such chambers is provided in WO 2019/244044, incorporated herein by reference. The use of organoids increases the accuracy of the cytotoxicity assessments because the organoids are more complex than the tissue strips and more closely mimic the in vivo organ, such as the heart. The organoid chambers in which the organoids are developed are also useful in conducting the cytotoxicity assessments of compounds because they are engineered to grow and maintain the organoids while providing for fluid (e.g., media) influx and fluid efflux (e.g., waste) in an environment equipped to allow for the controlled introduction of test compounds with the controlled electrical stimulation of the organoids. Moreover, the modular design of the organoid chambers facilitates the monitoring of multiple like, or unlike, organoids in a single housing wherein the monitoring is aided by the use of mirrors coordinated with monitoring and/or recording devices.

As noted, a single tier assay system, which may be a first-tier of a multi-tiered assay system, may comprise engineered tissue strips, such as human ventricular cardiac tissue strips (hvCTSs), that are useful for rapid determinations of compounds having cytotoxic effects. This first-tier screen may be combined with a second-tier screen of the compound using an organoid chamber, such as a human ventricular cardiac organoid chamber (hvCOC). The two-tiered screen for compounds having cytotoxic effects permits a staged focusing on compounds of interest while obtaining data on the effects of a compound on cells, e.g., cardiomyocytes such as human ventricular cardiomyocytes, found in the first-tier screen using an hvCTS, and combining that data with the effects of the compound on the higher order biological structure of a related organoid, such as a cardiac organoid, to confirm the findings of the first-tier screen and to reveal any organoid- or organ-level effects not apparent from the cells used in the first-tier screen. This approach not only allows for the rapid, progressive focus on compounds having cytotoxic effects, it assesses the effects of the compounds in two different, yet relevant, contexts, i.e., the cell-based context and the organoid- or organ-based context. Using this approach, the results are more reliable in being both more accurate and more reproducible than results obtained using any single-stage screen, such as the compound screening systems and methods currently in use. The disclosure further provides for the possibility of a third-tier screen optimally combined with the first- and second-tier screens.

In the third-tier screen, the organoids screen, e.g., the hvCOC organoid screen, is expanded to multiple organoids using a multi-organoid chamber system. The third tier screen yields data that can be even more reliable than the data obtained from two-tiered screening. In addition, the multi-organoid chamber format allows for multiple organoids of the same type, e.g., cardiac organoids, to be used in the screen and/or for different organoids to be used in the third-tier screen at the same time, using the versatile multi-organoid chamber system disclosed herein. (As used herein, “organoid” typically refers to an organ-like biomaterial, but the term can also refer to a tissue, which can be considered an organ-like biomaterial. The meaning of the term used herein will be apparent from the context of its usage.) The technology is versatile in being suited for the identification of compounds having cytotoxic effects. In some embodiments, the human ventricular cardiac tissues comprise hPSC-derived ventricular cardiomyocytes (VCMs). The single-cell properties of such cells, such as electrophysiology (action potential, Ca²⁺ handling), transcriptome, proteome, and the like, have been extensively characterized. As disclosed herein, various cells, e.g., human ventricular cardiomyocytes, may be used in developing the organoids used in the second-tier chamber system and in developing the organoids used in the third-tier multi-organoid chamber system.

In some embodiments, one or more organoid chambers may be provided in a bioreactor used to culture many, and in some cases a variety of, tissue-engineered human organoids. The device is designed to allow interconnection and simultaneous measurement of multiple organoids, with features that enhance reproducibility and efficiency in organoid function testing by enabling subsequent characterizations to be performed within the same bioreactor with minimal manipulation or intervention by the operator.

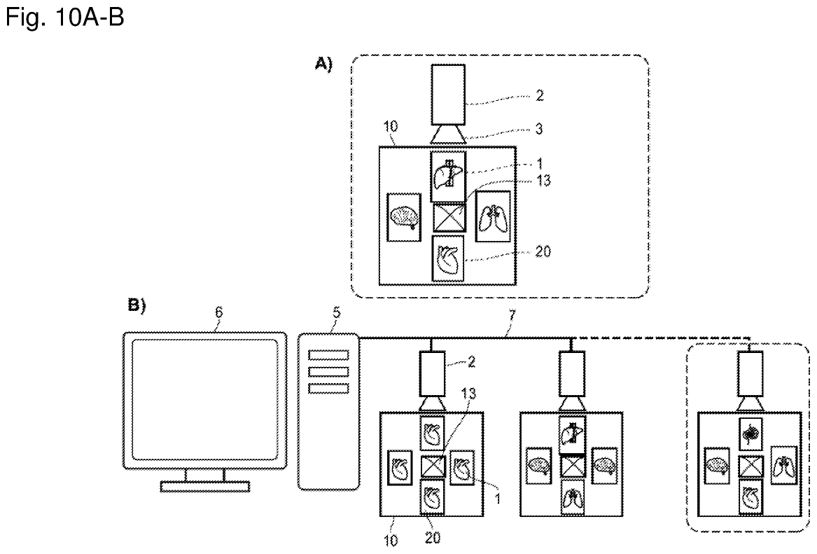

FIGS. 10A-B provides a high-level schematic view illustrating the versatility of the disclosed bioreactor system. FIG. 10A shows an organoid module 10 which contains at least one organoid cartridge 20. An organoid cartridge 20 contains a single organoid 1 of any type (e.g., heart, brain, nerve, liver, kidney, adrenal gland, stomach, pancreas, gall bladder, lung, small intestine, colon, bladder, prostate, uterus, blood, vascular, tumor, eye, or skin, and the like). An organoid module 10 may contain multiple organoid cartridges 20, and thus may contain multiples of a single type of organoid 1 or a variety of organoids 1. The organoid module 10 is oriented such that a detection/recording device 2, e.g., a camera, can detect and record the contents of the organoid module 10, such as by having a face of the organoid module 10 closest to detection/recording device 2, and preferably perpendicular to the device, be substantially or completely transparent to at least one wavelength of the electromagnetic spectrum detected by detection/recording device 2. FIG. 10B presents a data processor 5, e.g., a computer, in connection with at least one organoid module 10. The organoid modules 10 are typically in 1:1 correspondence with the detection/recording devices 2, and the detection/recording devices 2 are in electronic communication with data processor 5 via communication path 7, e.g., either conventional electrical wiring or by wireless communication. Video monitor 6 may also be connected to data processor 5 via communication path 7.

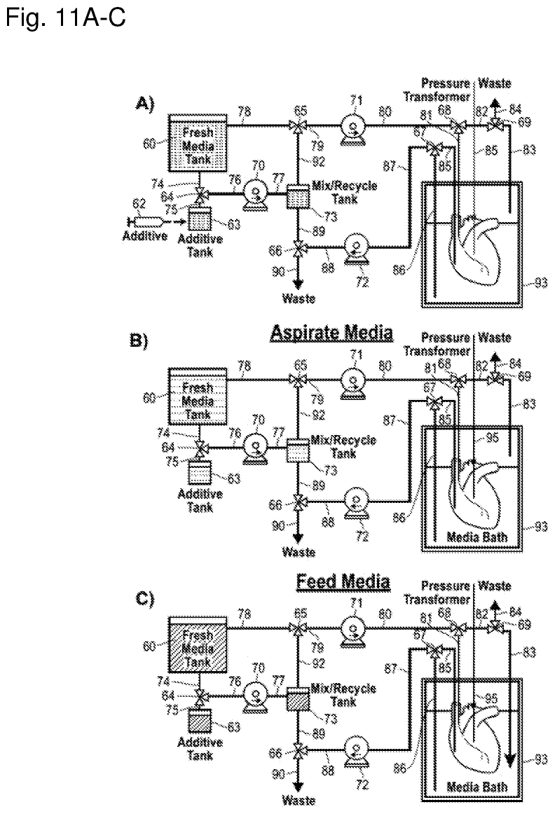

FIG. 11A-C illustrates elements of an embodiment of the bioreactor system that are involved in fluid movements, e.g., media flow, particularly the fluid movements involved in adding, or feeding, fresh media and removing, or aspirating, spent, or waste, media. FIG. 11A illustrates the entirety of the fluidic exchange system for a single organoid chamber in an organoid module, such as the single organoid chamber 20 in organoid module 10 of FIG. 10A. FIG. 11B provides the combination of activated valves and pumps for aspiration and FIG. 11C provides the combination for feeding fresh media. The components of the system involved in fluid, e.g., media, movements can be located within, or without, organoid module 10. In describing an embodiment of the bioreactor system providing fluid movements as illustrated in FIG. 11A-C, attention is focused on FIG. 11B for media aspiration and on FIG. 11C for feeding of media to cells, tissues and organoids of the disclosure. It is understood that the combined descriptions of aspiration and feeding will provide a description of the complete fluid communications within an embodiment of the bioreactor system, as illustrated in FIG. 11A. In the remaining description of FIGS. 11A-C, attachments are to be understood as providing fluid communication between the attached components.

Turning now to the features of FIG. 11B involved in one embodiment of the bioreactor system for aspirating media, media 93 is in contact with chamber media-junction D tubing 86, which is attached to junction D valve 67. Also attached to junction D valve 67 is organoid-junction D tubing 85. In addition, junction D valve 67 is attached to junction D-pump C tubing 87, which is attached to pump C 72. Pump C 72 is attached to pump C-junction C tubing 88, which is attached to junction C valve 66. Junction C valve 66 is attached to junction C-mix/recycle tank tubing 89, which in turn is attached to mix/recycle tank 73. In some embodiments, media 93 is recycled and routed towards mix/recycle tank 73. Junction C valve 66 is also attached to junction C-waste tubing 90 leading from junction C valve 66 to waste.

In operation, the feeding of cells of the organoid, involves components highlighted in FIG. 11C, including fresh media tank 60, which is attached to fresh media-junction B tubing 78, which in turn is attached to junction B valve 65. Junction B valve 65 is attached to junction B-pump B tubing 79, which is attached to pump B 71. Pump B 71 is in turn attached to pump B-junction E tubing 80, which is attached to junction E valve 68. Junction E valve 68 is also attached to junction E-junction F tubing 82, which is attached to junction F valve 69. Also attached to junction F valve 69 is chamber media-junction F tubing 83, which also contacts chamber media 93.

Additional attached components are described that provide fluid communication within the system and permit additional functions including, but not limited to, therapeutic additive dilution, perfusion of therapeutic(s), therapeutic washout of organoid, rinsing of fluidic lines, and the like. Fresh media tank 60 is attached to, and in fluid communication with, fresh media-junction A tubing 74, which in turn is attached to, and in fluid communication with, junction A valve 64, e.g., a three-way fluid controller or valve. Additive container 62, e.g., a therapeutic container, is used to deliver at least one therapeutic to additive tank 63, which is attached to additive tank-junction A tubing 75, which in turn is attached to junction A valve 64. Junction A valve 64 is also attached to junction A-pump A tubing 76, which is attached to pump A 70. Pump A 70 is attached to pump A-mix/recycle tank tubing 77, which is in turn attached to mix/recycle tank 73. In some embodiments, media from fresh media tank 60 is used to dilute therapeutic(s) from additive tank 63 within mix/recycle tank 73. Mix/recycle tank 73 is also attached to mix/recycle tank-junction B tubing 92, which in turn is attached to junction B valve 65. Junction E valve 68 is attached to junction E-organoid chamber tubing 81. In some embodiments, media can be delivered through junction E-organoid chamber tubing 81 to increase pressure within organoid 1. A pressure probe, i.e., pressure transducer 95, detects pressure, and changes in pressure, within an organoid 1 and converts the pressure to an analog electrical signal that is typically transmitted to the data processor, thereby allowing pressure to be monitored and adjusted by the system. In addition, the device provides for the washout or rinsing of fluidic lines. In particular, junction F valve 69 is attached to junction F-waste tubing 84, which in turn leads from junction F valve 69 to waste. In some embodiments, fluid can be removed from the fluid exchange system without coming in contact with organoid chamber 20 by exiting to waste through junction F-waste tubing 84.

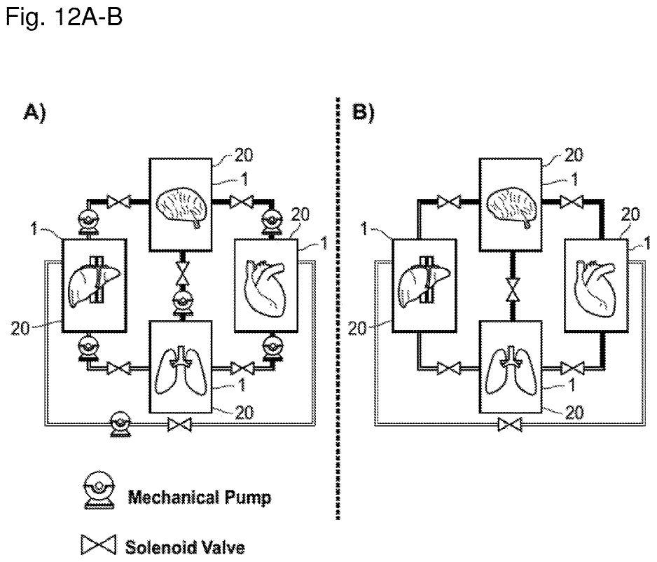

FIGS. 12A-B presents a higher-level schematic of fluidic exchange within organoid module 10 to illustrate the creation of a “body-in-a-jar”. FIG. 12A presents a fluidic exchange system that transfers media between at least two organoid chambers 20 within organoid module 10. Fluid is directed through the system by a series of valves and pumps. FIG. 12B illustrates a fluidic exchange system where fluid is directed by valves and is pumped solely by a biological pump (e.g., a heart organoid 1), thereby providing a self-powered “body-in-a-jar”

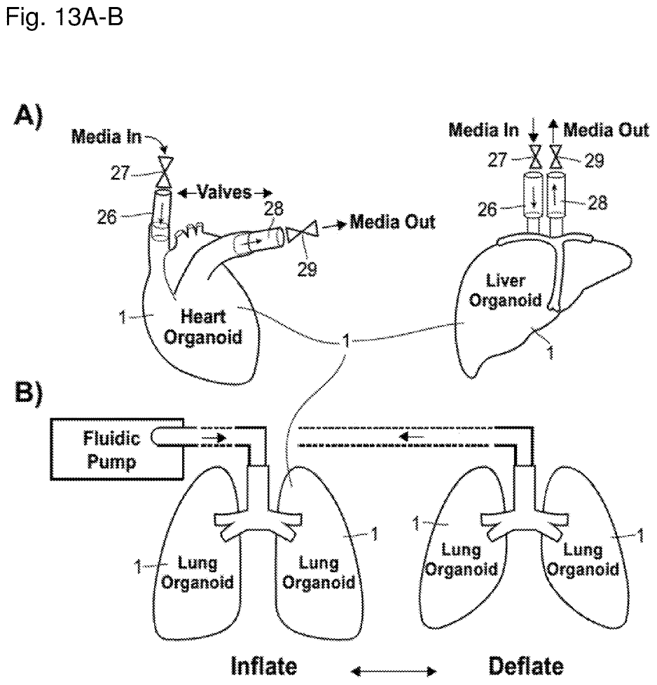

FIGS. 13A-B presents methods of flowing fluid into and out of organoids 1. FIG. 13A illustrates an organoid 1 (left panel: heart organoid; right panel: liver organoid) connected to media inlet tube 26 and media outlet tube 28, which permits fluid to be directed into the void of the organoid 1 and out through media outlet tube 28 to a waste path. The direction of fluid flowing through the organoid 1 is controlled by inlet valve 27 and outlet valve 29. FIG. 13B represents a method of applying mechanical pressure to an organoid 1, such as a lung organoid 1. A fluidic pump controls fluid flow (e.g., gas or liquid) to organoid 1 and modulates the pressure of the organoid cavity to control the size of organoid 1. Absolute pressure values depend on the material properties of the organoid 1 and the desired size of the membrane for a given application. Applied relative pressures are adjusted for mechanical strain up to 25%.

As would be apparent to those in the field, some features of the bioreactor system are optional and most of the features of the system exist in a variety of embodiments. In some embodiments, cells can be sourced from any mammalian species or engineered as organoids 1 from cells and extracellular matrix. Any organ tissue type is suitable for use in the disclosed system, compositions and methods. For example, tissues can act as surrogates for any organ, including but not limited to the heart, brain, nerve, liver, kidney, adrenal gland, stomach, pancreas, gall bladder, lung, small intestine, colon, bladder, prostate, uterus, blood, vascular, tumor, eye, and skin.

Organoid chamber 20 containing organoid 1 (see, e.g., FIG. 10B) is typically a cube made of a transparent solid that can be disposable or sterilizable, with at least two access ports such as doors. Suitable transparent solids include glass, and clear plastics such as polystyrene, acrylic and polycarbonate. Organoid chamber 20 can also be any polygonal shape provided that detection/recording device 2 can detect and record the behavior of cells in organoid 1 within organoid chamber 20. Given that the structure of organoid chamber 20 is limited by the need to allow detection/recording device 2 to detect cell behavior, it is apparent that a variety of transparent and translucent materials may be used in constructing organoid chamber 20. Even opaque materials are envisioned in embodiments where detection/recording device 2 is not detecting the transmission of visible light from organoid 1. Organoid chamber 20 is also constructed to be fluid-tight, thereby allowing organoid chamber 20 to contain chamber media 93 to feed the cells of organoid 1. Also, a chamber lid can provide apertures for penetration of at least one electrode or a pressure probe, i.e., pressure transducer 95.

At least one organoid chamber 20 is contained in an organoid module 10, which is formed from materials similar to the materials used for organoid chamber 20 (see, e.g., FIG. 9A). Organoid modules 10 are typically square or rectangular in plane view, and typically contain a top in addition to a bottom. Organoid modules 10 are sized to accommodate at least 1, 2, 3, 4, 5, 6, 8, 10, or more organoid chambers 20. The walls, top and bottom of organoid module 10 are typically formed of a transparent solid such as glass or a clear plastic (e.g., acrylic or polycarbonate), but may also be formed of translucent or opaque materials provided that detection/recording device 2 can detect, and record, cell behavior. Organoid module 10 also typically contains one or more light sources 12, and one or more mirrors 13, such as a pyramidal mirror 13. In several embodiments, there is at least one light source 12 and at least one surface of a mirror 13 for each organoid chamber 20 contained in an organoid module 10.

Remaining components of the system include tanks, such as fresh media tank 60, mix/recycle tank 73, and additive tank 63, which are vessels for containing fluids used in the system. Such tanks can be any of a variety of dimensions and made from any of a number of materials, provided that the tanks as constructed can be used in an environment designed to minimize biological contamination, such as a sterile environment, and provided that the material used is compatible with the creation of one or more ports for fluid movement. Embodiments of the system may also involve one or more pumps, such as pump A 70, pump B 71 and pump C 72 (see, e.g., FIG. 11), and such pumps can be the same or different and can operate on any principle known to provide for the movement of fluids such as air and/or media through tubes. Exemplary pumps include peristaltic pumps, siphon pumps compatible with sterile environments, positive-displacement pumps such as piston-driven pumps, and non-positive-displacement pumps such as centrifugal pumps. In some embodiments, gravity is used to move fluids and no pumps are used to move, e.g., media.

Organoid module 10 (see, e.g., FIG. 10) also can interface with various tubes as illustrated in FIG. 11 to move gas, such as air, used to provide pressure, e.g., inflate an organoid, which can be a balloon (e.g., a 6-Fr silicon Foley catheter balloon) or to move fluid. Pressure variations sufficient to control the inflation of a balloon or to move fluid in the system are achieved at pressures compatible with the use of a wide array of tube types and not just tubing certified to handle high pressure. For example, clear, plastic, flexible tubing is suitable for use, such as Tygon® tubing. Moreover, the various tubes can be combined into a single run of tubing as noted above, and such combined tubing is particularly well-suited for use with peristaltic pumps. Additionally, the tubing used in a given embodiment can vary in composition, internal diameter and external diameter. Another feature of the system are the junctions. Junctions typically are connected or attached to two or three tubes, which can vary in diameter and composition, as noted above. These junctions can be mere conduits or, more typically, are valves capable of directing the flow of fluid such as media from any attached tube to any other one or two attached tubes. Additional features and variations thereof will become apparent from the entirety of the disclosure provided herein.

In some embodiments, the organoid model has inflow and outflow fluid pathways (FIG. 11A). Valves (e.g., check valves, solenoid valves) control the direction of fluid movement in and out of an organoid with a cavity. In some embodiments, a single shaft or tube for inflow and outflow is contemplated (FIG. 11B). A fluidic pump controls fluid flow rate into and out of the organoid. In some embodiments, unequal inflow and outflow fluid rates are used to control the amount of fluid within the organoid. Adjusting the volume within the organoid cavity results in mechanical stretch in pliable organoids. In some embodiments, stretch is applied as a step function (passive stretch) or a sigmoidal function (cyclic stretch). Mechanical stretch is considered to be a mechanotransduction signal in many organoid types. In some embodiments, a combination of mechanical and electrical stimulation presents a more robust response for therapeutic screening.

A fluidic exchange system automates routine media changes, adjusts intraluminal pressure, perfuses therapeutics during screening, and exchanges media between organoids (FIGS. 10 and 11). The fluidic system consists of a series of microfluidic pumps, 3-way valves controlled by a digital output board, and media reservoirs. Changing the valve configuration alters the direction in which media travels. In some embodiments, fluid can be added to, or removed from, a hollow vertical mounting shaft to which an organoid is connected, thus adjusting the hydrostatic pressure. A pressure transducer 95 and signal conditioner (e.g., OPP-M and LifeSens) senses the mean pressure within the organoid and communicates with the pumps, e.g., via LabVIEW, to adjust for a desired intraluminal pressure. Additionally, the fluidic exchange system is used for the mixing and perfusion of therapeutics to the organoid. A solution is pumped from the additive tank and mixed with the circulating media. The therapeutic then perfuses into organoid chamber 20 and through organoid 1, similar to drug delivery via blood flow in humans. In some embodiments, the fluidic system of pumps and valves connects at least two organoid chambers 20 within an organoid module 10 to permit exchange of media and/or therapeutic(s) between or among the organoids 1. Additionally, in some embodiments the fluidic exchange system between organoid chambers 20 is powered by a biological pump in the form of an organoid 1, such as a cardiac organoid 1.

### Example 5

hvCTS Model of Heart Failure with Preserved Ejection Fraction (HFpEF)

To create an in vitro model of HFpEF, hvCTS myocardial tissue strips are engineered as described herein above for the fabrication of hvCTS, and cultured in DMEM supplemented with 10% NCS, with daily half-medium changes, for five days to allow tissue formation and compaction. To induce the HFpEF phenotype, hvCTS are then treated with TGF-β1 (1 ng/ml) for 4.5 days, followed by treatment with TGF-β1 (1 ng/ml) plus ET-1 (100 nM) for 1 additional day, prior to evaluation of contractile function as described herein above. This combined TGF-β1/ET-1 treatment causes a significant increase in hvCTS passive force and diastolic stiffness and slowing of contraction and relaxation kinetics, with no significant change in systolic or developed force, compared to untreated hvCTS controls, recapitulating some of the key phenotypic features of patients with HFpEF. Transcriptomic analysis of bulk RNAseq data from HFpEF-hvCTS showed patterns of differential gene expression similar to data from HFpEF patient heart tissue, and revealed down-regulation of the SERCA2a gene (ATP2A2) relative to their respective controls, thereby identifying restoration of SERCA2a as a target candidate for mitigating the HFpEF disease traits.

### Example 6

### Testing Efficacy of SUMOylation Activators and E1 Ligase Activators for Treating Human Heart Failure In Vitro

To investigate the efficacy of carboxamide compounds as SUMOylation activators/E1 Ligase activators for treating heart failure in a human-specific in vitro model system, the compounds are used to treat Novoheart's unique in vitro model of human HFpEF. The resulting effects on cardiac functional properties are compared to time-matched untreated HFpEF-hvCTS as negative controls, and compared to time-matched healthy hvCTS as positive controls. Based on the demonstrated mechanism of action of carboxamide compounds and their stereoisomers on cardiomyocyte contractility by activation of SERCA2a and restoring Ca cycling, treatment of HFpEF-hvCTS with SUMOylation activators and E1 Ligase activators reduces diastolic stiffness and hasten twitch force contraction and relaxation kinetics, compared to untreated HFpEF-hvCTS controls, returning the metrics of cardiac function back toward healthy hvCTS values and thereby rescuing key aspects of the HFpEF disease phenotype. Thus, SUMOylation activators/E1 Ligase activators enhance lusitropy in the setting of HFpEF.

### REFERENCES

1. Kho, C., et al., Small-molecule activation of SERCA2a SUMOylation for the treatment of heart failure. Nat Commun, 2015. 6: p. 7229.
2. Go, A. S., et al., Heart disease and stroke statistics—2014 update: a report from the American Heart Association. Circulation, 2014. 129 (3): p. e28-e292.
3. Ramani, G. V., P. A. Uber, and M. R. Mehra, Chronic heart failure: contemporary diagnosis and management. Mayo Clin Proc, 2010. 85 (2): p. 180-95.
4. Sakata, S., et al., Restoration of mechanical and energetic function in failing aortic-banded rat hearts by gene transfer of calcium cycling proteins. J Mol Cell Cardiol, 2007. 42 (4): p. 852-61.
5. Sakata, S., et al., Transcoronary gene transfer of SERCA2a increases coronary blood flow and decreases cardiomyocyte size in a type 2 diabetic rat model. Am J Physiol Heart Circ Physiol, 2007. 292 (2): p. H1204-7.
6. Hadri, L., et al., SERCA2a gene transfer enhances eNOS expression and activity in endothelial cells. Mol Ther, 2010. 18 (7): p. 1284-92.
7. Melchior, F., M. Schergaut, and A. Pichler, SUMO: ligases, isopeptidases and nuclear pores. Trends Biochem Sci, 2003. 28 (11): p. 612-8.
8. Hay, R. T., SUMO: a history of modification. Mol Cell, 2005. 18 (1): p. 1-12.
9. Meulmeester, E. and F. Melchior, Cell biology: SUMO. Nature, 2008. 452 (7188): p. 709-11.
10. Kho, C., et al., SUMO1-dependent modulation of SERCA2a in heart failure. Nature, 2011. 477 (7366): p. 601-5.
11. Tilemann, L., et al., SUMO-1 gene transfer improves cardiac function in a large-animal model of heart failure. Sci Transl Med, 2013. 5 (211): p. 211ra159.
12. Ahner, A. and R. A. Frizzell, SUMOylation Modulates CFTR Biogenesis: Is the Pathway Druggable? Curr Drug Targets, 2015. 16 (9): p. 965-75.
13. Licciardello, M. P. and S. Kubicek, Pharmacological treats for SUMO addicts. Pharmacol Res, 2016. 107: p. 390-397.
14. Woo, C. H. and J. Abe, SUMO—a post-translational modification with therapeutic potential? Curr Opin Pharmacol, 2010. 10 (2): p. 146-55.
15. Wang, J. and R. J. Schwartz, Sumoylation and regulation of cardiac gene expression. Circ Res, 2010. 107 (1): p. 19-29.
16. Cashman, T. J., et al., Construction of Defined Human Engineered Cardiac Tissues to Study Mechanisms of Cardiac Cell Therapy. J Vis Exp, 2016 (109): p. e53447.
17. Turnbull, I. C., et al., Advancing functional engineered cardiac tissues toward a preclinical model of human myocardium. FASEB J, 2014. 28 (2): p. 644-54.
18. Shah, S. J. et al. Phenotype-Specific Treatment of Heart Failure With Preserved Ejection Fraction: A Multiorgan Roadmap. Circulation 134, 73-90 (2016).
19. Borlaug, B. A., Sharma, K., Shah, S. J. & Ho, J. E. Heart Failure With Preserved Ejection Fraction: JACC Scientific Statement. J Am Coll Cardiol 81, 1810-1834 (2023).
20. Hahn, V. S. et al. Myocardial Gene Expression Signatures in Human Heart Failure With Preserved Ejection Fraction. Circulation 143, 120-134 (2021).
21. Sharma, K. & Kass, D. A. Heart failure with preserved ejection fraction: mechanisms, clinical features, and therapies. Circ Res 115, 79-96 (2014).
22. Chowdhury, M. A. et al. Endothelin 1 Is Associated with Heart Failure Hospitalization and Long-Term Mortality in Patients with Heart Failure with Preserved Ejection Fraction and Pulmonary Hypertension. Cardiology 143, 124-133 (2019).
23. Widyantoro, B. et al. Endothelial cell-derived endothelin-1 promotes cardiac fibrosis in diabetic hearts through stimulation of endothelial-to-mesenchymal transition. Circulation 121, 2407-2418 (2010).
24. Oberhaus, S. M. TUNEL and immunofluorescence double-labeling assay for apoptotic cells with specific antigen(s). Methods Mol Biol 218, 85-96 (2003).
25. Mishra, S. & Kass, D. A. Cellular and molecular pathobiology of heart failure with preserved ejection fraction. Nat Rev Cardiol 18, 400-423 (2021).
26. Dobaczewski, M., Chen, W. & Frangogiannis, N. G. Transforming growth factor (TGF)-beta signaling in cardiac remodeling. J Mol Cell Cardiol 51, 600-606 (2011).
27. Yousefi, F. et al. TGF-beta and WNT signaling pathways in cardiac fibrosis: non-coding RNAs come into focus. Cell Commun Signal 18, 87 (2020).
28. Hanna, A. & Frangogiannis, N. G. The Role of the TGF-beta Superfamily in Myocardial Infarction. Front Cardiovasc Med 6, 140 (2019).
29. Serrao, G. W. et al. Myocyte-depleted engineered cardiac tissues support therapeutic potential of mesenchymal stem cells. Tissue Eng Part A 18, 1322-1333 (2012).
30. Turnbull, I. C. et al. Advancing functional engineered cardiac tissues toward a preclinical model of human myocardium. FASEB J 28, 644-654 (2014).
31. Keung, W. et al. Human Cardiac Ventricular-Like Organoid Chambers and Tissue Strips From Pluripotent Stem Cells as a Two-Tiered Assay for Inotropic Responses. Clin Pharmacol Ther 106, 402-414 (2019).
32. Lee, E. K. et al. Machine Learning of Human Pluripotent Stem Cell-Derived Engineered Cardiac Tissue Contractility for Automated Drug Classification. Stem Cell Reports 9, 1560-1572 (2017).

All patents and other publications identified are expressly incorporated herein by reference in their entireties or in relevant part, as would be apparent to the skilled person from the context, for the purpose of describing and disclosing, for example, the methodologies described in such publications that might be used in connection with information described herein.

What is claimed is:

1. A method of treating heart disease comprising administering a therapeutically effective amount of a carboxamide derivative compound to a patient with heart disease, wherein the carboxamide derivative compound induces increased SUMOylation of sarcoplasmic reticulum calcium ATPase type 2a.

2. The method of claim 1 wherein the carboxamide derivative compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide).

3. The method of claim 1 wherein the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide).

4. The method of claim 3 wherein the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide).

5. The method of claim 1 wherein the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

6. The method of any one of claims 1-5 wherein the heart disease is heart failure, cardiomyopathy, diastolic dysfunction, or muscular dystrophy.

7. The method of claim 6 wherein the heart failure is pressure overload-induced heart failure or ischemic heart failure.

8. A method of inducing a positive inotropic effect and/or lusitropic effect in a cardiomyocyte tissue by contacting the cardiomyocyte with a carboxamide derivative compound.

9. The method of claim 8 wherein the carboxamide derivative compound induces increased SUMOylation and/or increased E1 ligase activation of sarcoplasmic reticulum calcium ATPase type 2a.

10. The method of claim 8 or 9 wherein the compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide).

11. The method of claim 8 or 9 wherein the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide).

12. The method of claim 11 wherein the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide).

13. The method of claim 8 or 9 wherein the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

14. The method of any one of claims 8-13 wherein the positive inotropic effect and/or lusitropic effect is increased force of cardiac contraction, improved cardiac contraction, improved relaxation kinetics, and/or increased frequency of beating of the cardiomyocyte tissue.

15. The method of claim 8-13 wherein the positive inotropic effect and/or lusitropic effect is increased contractile force, heartbeat frequency, and/or heartbeat dependent contractile force.

16. A method of screening for a compound inducing an increase in contractile force, relaxation kinetics, heartbeat frequency, and/or heartbeat dependent contractile force in a human cardiomyocyte tissue comprising
(a) contacting a cardiomyocyte tissue with at least one positive amount of the compound;
(b) electrically stimulating the tissue;
(c) measuring the contractile force, relaxation kinetics, heartbeat frequency, and/or heartbeat dependent contractile force of the cardiomyocyte tissue;
(d) comparing the contractile force, relaxation kinetics, heartbeat frequency, and/or heartbeat dependent contractile force measured in (c) to the same property or properties of contractile force, relaxation kinetics, heartbeat frequency, and/or heartbeat dependent contractile force in a control cardiomyocyte tissue not contacted by the compound; and
(e) identifying the compound as a compound inducing an increase in contractile force, relaxation kinetics, heartbeat frequency, and/or heartbeat dependent contractile force in a human cardiomyocyte if the contractile force, relaxation kinetics, heartbeat frequency, and/or heartbeat dependent contractile force increases with increasing amount of compound.

17. The method of claim 16 wherein the compound is a carboxamide derivative compound.

18. The method of claim 17 wherein the carboxamide derivative compound induces increased SUMOylation and/or E1 ligase activation of sarcoplasmic reticulum calcium ATPase type 2a.

19. The method of any one of claims 16-18 wherein the cardiomyocyte tissue is a mature human ventricular cardiomyocyte tissue strip.

20. The method of any one of claims 16-19 wherein the compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide).

21. The method of any one of claims 16-19 wherein the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide).

22. The method of claim 21 wherein the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide).

23. The method of any one of claims 16-19 wherein the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

24. A method of increasing the activity of at least one small ubiquitin-like modifier type 1 polypeptide in a cardiomyocyte comprising administering an effective amount of a carboxamide derivative compound.

25. The method of claim 24 wherein the carboxamide derivative compound induces increased SUMOylation and/or E1 ligase activation of sarcoplasmic reticulum calcium ATPase type 2a.

26. The method of claim 24 or 25 wherein the carboxamide derivative compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide).

27. The method of claim 24 or 25 wherein the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide).

28. The method of claim 27 wherein the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide).

29. The method of claim 24 or 25 wherein the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

30. A method of increasing the activity of the sarcoplasmic reticulum calcium ATPase type 2a protein in a cardiomyocyte comprising administering an effective amount of a carboxamide derivative compound.

31. The method of claim 30 wherein the carboxamide derivative compound induces increased SUMOylation and/or E1 ligase activation of sarcoplasmic reticulum calcium ATPase type 2a.

32. The method of claim 30 or 31 wherein the carboxamide derivative compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide).

33. The method of claim 30 or 31 wherein the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide).

34. The method of claim 33 wherein the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide).

35. The method of claim 30 or 31 wherein the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).

36. A method of increasing the activity of E1 ligase in a cardiomyocyte comprising contacting the cardiomyocyte with an effective amount of a carboxamide derivative compound.

37. The method of claim 36 wherein the carboxamide derivative compound induces increased SUMOylation and/or E1 ligase activation of sarcoplasmic reticulum calcium ATPase type 2a.

38. The method of claim 36 or 37 wherein the carboxamide derivative compound is compound 6 (1-(4-benzoylphenyl)-5-oxo-N-(1-(pyrazolo[1,5-a]pyridin-3-yl)ethyl) pyrrolidine-3-carboxamide), compound 9 (5-oxo-N-(1-(pyrazolo[1,5-a]pyridin-3-yl)ethyl)-1-(pyridin-3-yl) pyrrolidine-3-carboxamide), or compound 11 (5-oxo-N-(1-(pyrazolo[1,5-a]pyridin-3-yl)ethyl)-1-(quinolin-3-yl) pyrrolidine-3-carboxamide).

39. The method of claim 36 or 37 wherein the compound is compound 6-1 (1-(4-benzoylphenyl)-(R)-5-oxo-N—((R)-1-(pyrazolo[1,5-a]pyridin-3-yl)ethyl) pyrrolidine-3-carboxamide), compound 6-2 (1-(4-benzoylphenyl)-(S)-5-oxo-N—((S)-1-(pyrazolo[1,5-a]pyridin-3-yl)ethyl) pyrrolidine-3-carboxamide), or compound 9-3 ((R)-5-oxo-N—((S)-1-(pyrazolo[1,5-a]pyridin-3-yl)ethyl)-1-(pyridin-3-yl) pyrrolidine-3-carboxamide).

40. The method of claim 39 wherein the carboxamide derivative compound is compound 6-1 (1-(4-benzoylphenyl)-(R)-5-oxo-N—((R)-1-(pyrazolo[1,5-a]pyridin-3-yl)ethyl) pyrrolidine-3-carboxamide) or compound 6-2 (1-(4-benzoylphenyl)-(S)-5-oxo-N—((S)-1-(pyrazolo[1,5-a]pyridin-3-yl)ethyl) pyrrolidine-3-carboxamide).

41. The method of claim 36 or 37 wherein the carboxamide derivative compound is compound 9-1 ((R)-5-oxo-N—((R)-1-(pyrazolo[1,5-a]pyridin-3-yl)ethyl)-1-(pyridin-3-yl) pyrrolidine-3-carboxamide), compound 9-2 ((S)-5-oxo-N—((S)-1-(pyrazolo[1,5-a]pyridin-3-yl)ethyl)-1-(pyridin-3-yl) pyrrolidine-3-carboxamide), compound 9-3 ((R)-5-oxo-N—((S)-1-(pyrazolo[1,5-a]pyridin-3-yl)ethyl)-1-(pyridin-3-yl) pyrrolidine-3-carboxamide), or compound 9-4 ((S)-5-oxo-N—((R)-1-(pyrazolo[1,5-a]pyridin-3-yl)ethyl)-1-(pyridin-3-yl) pyrrolidine-3-carboxamide).
